(19) United States
(12) Patent Application Publication
(10) Pub. No.: US 2022/0401027 A1
(43) Pub. Date: Dec. 22, 2022

(54) Forward-Looking Precision Imaging Surgical Probe

(71) Applicant: NeurOCTix, El Macero, CA (US)

(72) Inventors: Thomas Mowery, El Macero, CA (US); John Black, Bainbridge Island, WA (US); David Smith, Redwood City, CA (US); Kenneth A. Murray, JR., Davis, CA (US)

(73) Assignee: NeurOCTix, El Macero, CA (US)

(21) Appl. No.: 17/845,799

(22) Filed: Jun. 21, 2022

Related U.S. Application Data
(60) Provisional application No. 63/213,121, filed on Jun. 21, 2021.

(51) Int. Cl. A61B 5/00

(52) U.S. Cl. CPC A61B 5/6848; A61B 5/0066; A61B 5/0084; A61M 25/065

(57) ABSTRACT

A precision forward-looking image-guided diagnostic and therapeutic surgical probe and needle insert for microsurgery in support of imagery, neurology, neurosurgical procedures, and ophthalmic surgical applications comprising an introducer needle (stylet), a fiber carrier, a therapeutic conduit, and a spirographic method for scanning a target and associated algorithms to create and render a reconstructed image for display to a physician in real-time or near real-time. The probe implements Optical Coherence Tomography (OCT) to provide high-resolution extended imagery of an intended therapeutic or target tissue. A separate therapeutic conduit provides surgical access for therapeutic devices such as a cutting or ablation laser, an RF electrode for locally heating tissue, a lumen for local injection of neurolytics/paralytics, placement of electrodes for neuromodulation, and deployment of a micro-endoscopic imaging tool. A third working channel supports the delivery of neurolytic and other fluids.

### CROSS-REFERENCE TO RELATED APPLICATIONS

This patent application claims the benefit of U.S. Prov. Ser. No. 63/213,121, filed Jun. 21, 2021.

### STATEMENT REGARDING FEDERALLY SPONSORED RESEARCH OR DEVELOPMENT

Not applicable

### FIELD OF THE INVENTION

The invention relates to internal visualization and treatment systems for surgery. In particular, the invention relates to probes having imaging inserts for incorporation with surgical tools.

### BACKGROUND

The evolution of medical imaging technology has reduced the need for open surgical procedures to determine medical issues. Additionally, many medical procedures previously executed blindly now benefit from guidance with image confirmation acquired through devices such as endoscopic probes having image-enabled catheters and needle probes. The advantages of image-enabled minimally invasive surgery include reduced cost, lower mortality and morbidity, reduced hospital admissions, and enhanced patient satisfaction. One evolving imaging technology is Optical Coherence Tomography (OCT). OCT can be used independently or in conjunction with other imaging modalities such as Mill, CT, Ultrasound, and X-ray.

Optical coherence tomography (OCT) is an imaging technique that uses low-coherence light to capture micrometer-resolution, two- and three-dimensional images from within optical scattering media (e.g., biological tissue). In one sense, OCT may be considered the optical analog of ultrasound but differs by the provision of higher resolution microscopic images, but with a more limited depth of penetration in tissue. Consequently, OCT has been extraordinarily successful in use with near surface-oriented medical imaging applications, including the assessment of surfaces of the skin and on internal luminal surfaces such as the gastrointestinal (GI) tract and coronary arteries. Surface applications of OCT are elegant but rely on large format devices that are not suitable for deep tissue intervention.

Technology has evolved to enable the miniaturization and successful commercialization of “side-looking” OCT surgical probes that enable intraluminal and canal imaging. However, “side-looking” OCT probes fail to avoid collateral damage caused by the advancing surgical instrument to deep tissues. They fail to identify critical anatomy, such as nerves and blood vessels, which can be present in the path of the surgical instrument, such as a cutting tool.

In contrast to a side-looking OCT probe, the macro-mechanics required for a forward-looking miniature OCT probe for deep tissue access have hampered the ability to develop clinically relevant surgical probes suitable for commercialization. One of the major challenges of deploying a desirably small forward-looking deep tissue surgical probe is that the field of view ahead of the probe narrows as the diameter of the probe lens narrows. Thus, the advantage of the forward-looking view diminishes, along with associated clinical relevance. It would be highly desirable to have a clinically relevant enface (forward-looking) OCT needle probe for deep tissue imaging, which is user-friendly, small diameter, has microscopic resolution, and is gentle to tissue while providing a significantly superior field of view. Applications for such a device would include, but not be limited to, needle biopsy for cancer, ablation of cancer with margin detection, fluorescence imaging, nerve blocks for reginal anesthesia, and epidural and spinal canal access along with others. Two additional procedures are described in detail to demonstrate the value of the enface needle probes.

### SUMMARY

An imaging probe designed to provide forward-looking precision-guided neurosurgery and ophthalmic procedures. The probe may be used to facilitate various treatments including but not limited to, nerve ablation, precision injection, electrode placement, and extraction of intra-ocular shrapnel. The probe uses dual rotational movement of imaging components to support forward-looking high-resolution imagery with a clinically relevant field of view. The imaging probe comprises a means for variable rotation speeds resulting in a spirographic imaging scanning pattern at variable densities to create a forward-looking image with enhanced areal coverage. This approach likewise provides a 3-dimensional volumetric view as well, in real-time and near real-time. The probe includes computer processing that executes logical instructions according to one or more interpolation and reconstruction algorithms to generate relevant images and volumetric assessments of various tissues useful to a physician/surgeon.

One complementary forward-looking minimally invasive application of OCT according to the present invention is the application of neuroablation, also known as radiofrequency ablation therapy (or RFA) for pain control and other medical objectives. Historically, numerous techniques have been used to selectively destroy nerve tissue in the brain, spine, and elsewhere in the body. These include cryogenic surgery, focused ultrasound, chemical destruction, ionizing radiation, mechanical methods, lasers, radiofrequency heating, and direct current heating. Of these approaches, radiofrequency (RF) and direct current heating methods involve the passage of electrical current from an electrode to the target tissue, thereby heating and destroying the tissue within a volumetric area in the vicinity of the electrode. Temperature is recognized as the fundamental lesion parameter. Unfortunately, in all these approaches, there is little if any ability to actually visualize the tissue being treated and monitor changes to the properties of the tissue during treatment.

The present invention is particularly suited to visualization of tissue during radiofrequency ablation treatments. Since its original development in the early 1900′s, radiofrequency treatment of various tissues has evolved to become one of the most effective and widely used techniques for selective destructive and non-destructive treatment of several types of tissue. Typically, narrow-gauge insulated electrode needles with a variable amount of non-insulated needle tip are placed either adjacent to or into a target tissue, typically using fluoroscopy for indirect external imaging. The electrode needles, connected to an energy generation device, deliver high-frequency radio waves above 250 kilocycles to the target tissues. Typical frequencies are in the range between 500 kHz and 1000 kHz. The energy flows from the electrodes through the target tissue; resistance to the energy flow creates heat within the target tissue. Typically, tissue temperatures above 44 degrees Centigrade will cause destruction to nerve tissue; tissue temperatures below 44 degrees Centigrade will not cause nerve tissue destruction. Pulsed applications of RF energy may be used to maintain tissue temperature below a target level to avoid widespread tissue damage. This approach may provide temporary relief, but full and lasting benefit may not be achieved. During this process, the physician is relying on prior experience to assess whether the radio frequency treatment is being properly and effectively applied

The radiofrequency energy delivery device can operate in either a monopolar or bipolar manner. In monopolar operation, energy flows to a dispersing grounding pad located on the exterior of the individual being treated. In bipolar operation, energy flows to an adjacent needle creating a controlled delivery of heat to the tissue within the target area or field of the two needles. The RF current heats the tissue, and the tissue heats the electrode tips, allowing accurate monitoring of tissue temperature. However, measuring tissue temperature is still an indirect method of determining the level of tissue ablation. This indirect approach is not conclusive as to determining the level and degree of tissue ablation.

According to the inventive subject matter, assessing the progress of the creation of a lesion can be accomplished using the described optical imaging techniques. Temperature sensing methodologies are unable to account for the heat which may develop in surrounding tissues, e.g., bone, creating an additional thermal reservoir of heat, which will affect time constants and complicate treatment procedures and the interpretation of success or failure. Consequently, using direct visual imaging of the target tissue using the present invention is essential to improving effectiveness of the treatment.

According to the inventive subject matter, electrode tips of about 1.1 mm in diameter and 3 to 5 mm in length can produce lesions of about 3 mm diameter and 4 to 7 mm in length when temperatures in the target tissue are 65 to 75 degrees Centigrade. Further, according to the present invention, exceptionally fine gauge electrodes of 0.25 mm (31 gauge) in diameter with 2 mm long tips will give rise to lesion sizes on the order of between 0.7 mm to 0.9 mm in diameter and 1.8 to 2.2 mm when tissues reach 75 degrees Centigrade and are held at that temperature for 15 seconds. Radiofrequency lesioning technology has been used in many medical therapy applications including heart rhythm control, snoring abatement, cancer tumor ablation, and pain management. Within the field of pain management, radiofrequency ablation has been used to create lesions in spinal cords, dorsal root ganglia, sympathetic nerves, small nerves to both large and small joints (such as the sacroiliac and facet joints), neuromas, feet, and vertebral column discs. The lesions created in the target nerve tissues interrupt pain signals from the targeted areas, frequently providing relief from chronic or acute pain.

Traditionally, spinal radiofrequency ablation has been applied in a monopolar manner using single, non-insulated needle tips from 4 to 10 millimeters long. Introducer needles of 18, 20 and 22 gauge are typically used for delivery of the electrode to a target site. For clarification, an 18-gauge needle has an outer diameter of 1.27 mm; a 20-gauge needle has an outer diameter of 0.902 mm; a 22-gauge needle has an outer diameter of 0.711 mm; a 25-gauge needle has an outer diameter of 0.508 mm. The narrower 25-gauge needle is typically not used for electrode delivery because it tends to be too flimsy to effectively percutaneously penetrate tissue.

A single needle ablation approach can create lesions designed to interrupt the flow of pain signals along targeted nerve tissue along the spinal column. However, even an experienced physician deals with the challenge of confidently placing a single needle electrode tip adjacent to a target nerve(s) due to several factors including (1) the geometric complexity of the spinal column, (2) the variable locations of nerves, (3) the variable and intricate paths of nerves, and (4) differences from person to person. Importantly, the target nerves are effectively invisible during the ablation procedure. Hence, the inclusion of high resolution in situ imaging in conjunction with the ablation procedure is invaluable.

For maximum effectiveness in single needle RF ablation applications, according to the present invention, it is desirable to position the single needle so that it lies parallel to the longitudinal axis of the targeted nerve fiber to ablate a long segment of the nerve as possible. Hence, it is important to implement a method that causes the needle to be placed in close proximity to the target nerve. Unfortunately, the structural complexity of the spine, an inability to know the exact location of a targeted nerve, and the variable location of targeted nerves can limit the ability of a physician to manipulate and align a single needle in parallel with the desired targeted nerve fiber. Consequently, without the benefits of additional imaging according to the present invention, the physician typically must make multiple placements of the single needle to ensure that a targeted nerve fiber is sufficiently covered by lesions to sufficiently ablate the target nerves to treat a patient's pain. To compensate for the shortcoming of single needle profile applications, newer needles have been developed that have a “fan” like active tip having multiple tines. The tines are intended to cover more area without having to reposition the needle. Without additional imaging capability according to the present inventions, one cannot be assured that the use of a fanned tine configuration adds substantial benefit in terms of outcomes.

Importantly, in either method, these “blind” approaches are likely to cause collateral damage to other surrounding tissue, creating undesirable and potentially severe side effects such as bleeding, increased postoperative pain, and prolonged recovery times. Patients frequently require strong pain medication post-operation, and occasionally patients will go to the emergency room post-operation for pain control. Moreover, neither the single needle nor the tine fan design alone permits target-specific access, real-time visualization of the ablation process, or assured confirmation of the success of the ablation procedure.

Single-needle RF ablation can be effective for applications such as spinal cord lesions. However, in most other spinal applications, a single needle RF ablation application is difficult to use and will not ensure adequate treatment at the target site. Without multiple needle placements, the single-needle electrode methodology is less effective for treating sympathetic ganglia, dorsal root ganglia, medial branch nerves to facets, and peripheral nerves, among others. For patient comfort and to expedite treatment, it is very desirable to have a system and apparatus to provide ablation and treatment of spinal nerve tissue while minimizing the number of needle repositioned placements required to create an effective lesion.

The objective of RF ablation treatment in pain management is to affect pain fibers (which are nerve fibers known to conduct pain signals) in such a way as to interrupt the transmission of pain signals from peripheral anatomy to the central nervous system. Such signal transmission interruption typically will cause the pain experienced by the subject to lessen significantly. Radiofrequency ablation treatment includes two primary approaches. The first, RF ablation, is a destructive methodology. The second, pulsed radiofrequency ablation, is considered a nondestructive treatment. A lesion, or destruction, of nerve tissue, occurs above 44 degrees centigrade. A lesion produces a total disruption of sensory conduction. Pulsed RF produces a partial interruption of the sensory conduction. The effects of RF ablation are both time and temperature dependent.

Terms such as lesion, ablation, neurolysis, and neurotomy are often used synonymously. Some chronic pain syndromes that may be treated by RF ablation include CRPS, cervicogenic headaches, trigeminal neuralgia, occipital headaches, cancer pain, neck pain, low back pain, chest wall pain, post herniorrhaphy pain, sacroiliac joint pain, foot pain, and facet-mediated pain. Facet mediated pain is well documented to occur especially after a whiplash injury. In addition, facet-mediated pain is frequently associated with arthritis to the facet joints

For the successful application of radiofrequency technology to the treatment of spinal pain, several steps are required. First, one must determine the peripheral source of the pain, such as a facet joint. Targeted injection of local anesthetics without spread to multiple tissues assists in the accurate diagnosis and identification of the peripheral pain source. If an anesthetic is injected at the location of a suspect pain fiber, the pain sensed by the patient should subside to a degree noticeable to the patient.

Once the fiber generating the peripheral source of pain has been diagnostically identified and can now be targeted, a physician will prepare a plan and procedure for attempting to safely direct a percutaneous RF delivery device, i.e., a needle, to be contiguous to the target nerve. In single needle delivery systems, alignment of the single needle in close proximity to the target nerve is essential to successful outcomes since the size of the generated lesion is limited by the size of the needle. Unfortunately, every treatment is still the result of an educated guess by the physician since the actual nerve fiber cannot be imaged during the treatment.

Access by the physician to the target site is dictated by the anatomical position of the target nerve fiber. Traditionally, in the treatment of spinal pain, a curved needle, usually 22, 20, or 18 gauge, facilitates directional placement of the needles. Ionizing X-ray fluoroscopy is used for imaging to assist in needle placement. However, multiple two-dimensional fluoroscopic views are typically necessary to increase the potential for accurate needle placement.

Consequently, in existing treatment methods, a repetitive pattern of directing and repositioning the needle using fluoroscopic X-ray imaging is required. In addition, frequent repositioning of the patient for adequate visualization using fluoroscopy may be required. These repetitive patterns tend to increase patient discomfort, increase the potential for harmful needle placement and extend the time and consequently the exposure to harmful X-rays required to provide the treatment.

An often overlooked negative also associated with this approach is that the treating physician, dealing with a number of patients, will normally suffer a much higher cumulative x-ray exposure from the fluoroscopy than any individual patient. Consequently, there is a high likelihood of negative future consequences for the physician which go unaddressed. Hence, it would be highly desirable to have a system or device that could limit X-ray exposure to both the patient and the physician.

Currently available pain management treatment systems and methods can be quite uncomfortable, sporadically effective, and very costly. A patient can be subjected to several hours of applied repetitive, uncomfortable procedures. The prohibitive cost along with limited effectiveness of the treatment, constrains availability of these treatments to a limited portion of the affected patient population. Thus, although potentially beneficial, the larger sector of the population having a need for the procedures does not have access. Consequently, there are millions of people around the world unnecessarily suffering. The cost and previously limited effectiveness due to the lack of the imaging technology according to the present invention, likewise, impedes a physician's ability to obtain approval for payment for the procedures from insurance providers, which further limits access.

Consequently, the application of these radio frequency ablation and other similar procedures in concert with the inventive subject matter, providing single-user, direct, microscopic real-time visualization of needle position and navigation, without dangerous X-ray exposure from fluoroscopy, is a highly desirable, but previously unavailable, technological capability. Three-dimensional visualization is extremely beneficial, but unavailable. Hence, the application of capabilities described according to the inventive subject matter would optimize and improve the treatment procedures, improve the quality of care, and provide confidence for the treating physician, the treated patient, and the payor to support the application of these treatments for the mutual benefit of millions of people just in the United States and even more around the world.

An aspect of the procedures associated with the inventive subject matter depends on these innovative approaches. For example, once target nerves are presumed to be located, and a needle is estimated to be placed contiguous to the nerves, a radio frequency generator can be used to deliver a measured band of radio waves to cause the target tissue to reach the desired temperature between 75 to 90 degrees Centigrade for a duration of 60 to 90 seconds with the intent to create a discrete destructive lesion, hopefully transecting the target nerve/tissue. Alternatively, the target tissue may be raised to a temperature of approximately 45 degrees Centigrade for a minimum of 120 seconds to implement a non-destructive treatment.

Various patents describe devices and methods for ablating tissue at various anatomical sites using radiofrequency energy. Others describe in greater detail variations in the design and operation of needles associated with delivering one or more electrodes of some type to a target ablation site.

Based on the limitations described in the prior art, an accurate ablation device would be highly desirable. It would be further desirable that such a device be able to provide confidence and confirmation in achieving successful ablation of nerves associated with spinal pain according to the various methods and at the plurality of locations described herein.

In one instance, this recognized need has encouraged the increased use of non-ionizing ultrasound as an alternative approach to fluoroscopy. However, ultrasound is exceedingly difficult to learn and requires a two-position view alternating between axial and cross-section. This usually requires two people; one to watch the patient and the other to use both hands to manipulate the ultrasound transducer apart from manipulating the expensive echogenic needle apparatus intended to visualize the needle tip. Further, the use of ultrasound transducers by multiple personnel at various locations creates a risk for inadequate sterilization and therefore an increased risk of potential infection. Additionally, the ultrasound images are vague and not anatomically obvious since they lack microscopic resolution.

As with existing ablation procedures, surgical procedures associated with the nervous system, the eye, the ears, and other areas of the body frequently suffer from the lack of a convenient and compact imaging methodology. It is particularly problematic when a targeted area is so small that standard surgical microscopic vision techniques and ultrasound do not provide sufficient resolution or accuracy.

In another detailed application, ophthalmic “cold steel” surgical procedures are normally performed with the aid of an ophthalmic surgical microscope using free-space stereoscopic optical visualization through the cornea to the lens, iris, and then through to the vitreous humor, and retina when posterior chamber work is done. Surgical tools are inserted through the cornea or sclera behind the iris and capsule when performing an anterior or posterior chamber procedure respectively, for example in cataract, vitrectomy, and retinal detachment surgery. These techniques are well established and have well-understood risk profiles; several million cataract surgeries are successfully performed in the United States every year.

A minimally invasive application of OCT involves avoidance of damaged corneas to extract shrapnel after blast injuries. Ophthalmic battlefield injuries can be (a) multi-focal, with multiple lacerations to the cornea and sclera, (b) retinal detachment often accompanied by inflammation, (c) proliferative vitreoretinopathy, (d) scar tissue formation, and/or (e) partial healing with incorrect wound margin apposition. Complications are amplified when a surgeon encounters a patient with an opacified or damaged cornea. The damaged cornea can interfere with standard visualization of the ophthalmic surgical field, which in turn restricts options for surgery. Interfering corneal damage can be caused by penetrating trauma (glass, shrapnel, etc.) and shock wave barotrauma. In addition, corneal damage can result from photochemical/thermal/chemical modification of the corneal/air interface. This type of corneal damage can be caused by exposure to, for example, chemical agents, especially nitrogen/sulfur mustards and chlorine gas, and/or, being near deflagrating heat sources (IEDs, propellants, phosphorus, etc.). Parenthetically, exposure to IEDs can cause a multiplicity of trauma sites to a solider, many of which can involve nerve fiber trauma, resulting in both acute and chronic pain, along with ophthalmic injuries.

One approach used in the case of an impaired trans-corneal visual path is to remove a small section of the damaged cornea by trephine, followed by the implantation of a temporary acrylic corneal plug in the trephined area, followed by standard surgical techniques as far as possible. Keratoprosthesis is a surgical procedure where a diseased or damaged cornea is replaced with an artificial cornea. The keratoprosthesis acts as a placeholder for a subsequent corneal transplant following successful healing of the more sight-threatening injuries. Depending on the elapsed time between the injury and the initial operation, and on the nature of the injury, the level of inflammation and innate healing response in the eye tissues is often such that the temporary keratoprosthesis will not “take” or hold. Rejection of the keratoprosthetics can be accompanied by severe, sometimes irreparable damage to the peri-surgical site so that any subsequent attempt to transplant a real cornea is likely be unsuccessful.

There exists a pressing need for minimally invasive surgical approaches to preserve corneal viability while allowing critical posterior chamber procedures to be performed safely and effectively. In addition, there is a pressing need for devices to enhance surgical visualization where a confined field of view limits standard visualization techniques, e.g., for nerve ablation.

In one embodiment, the probe allows effective use of Optical Coherence Tomography (OCT) to provide forward-looking high-resolution imagery in a compact hypodermic needle configuration. In one instance, the probe provides scanned OCT with a voxel resolution of approximately twenty μm (Z-propagation axis)×50 μm (X)×50 μm (Y) in a forward-looking cone with a full angle of the order of 60-70 degrees and with the cone apex at the needle tip. A second working channel of the probe, in addition to the imaging fiber channel, is provided to accommodate the application of various therapeutic devices, for example, a cutting or ablation laser, an RF electrode for locally heating tissue, or a lumen for local injection of drugs/chemicals (e.g., neurolytics/paralytics), support matrices containing stem cells, or tissue scaffolding to promote tissue regeneration.

The probe facilitates sensitive surgical applications where reducing the complexity, size and weight of the surgeon's tools provides multiple benefits. The surgeon can use the probe effectively and safely, for example, around a patient's eye without interfering with surgical workflow or introducing additional complications.

A complementary second working channel of the probe is adaptable to multiple uses. For example, the channel can be used to deploy a second imaging modality (visible light video imaging), or an ophthalmic therapeutic tool. The second working channel can support photocoagulation, a vitrectomy/cutting laser, local injection of anti-inflammatory drugs, injection and activation of tissue welding/soldering agents, placement of fiducial markers, and placement of contrast agents to aid other surgical procedures. In particular, the second working channel can serve as a conduit to deploy micro-endoscopic imaging to complement the OCT imaging feature of the probe. In this instance, a coherent fiber optic bundle provides a differentiated view of the tissue to be imaged. The second working channel will also allow the introduction of contrast agents to a tissue area to support image refinement.

In the ophthalmic context, the probe allows the surgeon to bypass the patient's cornea as the only visualization path for anterior/posterior chamber surgery. The surgeon can perform desired procedures and insert precision-guided surgical tools with onboard visualization into the anterior or posterior chambers without requiring visualization via the cornea. These precision-guided surgical tools, leveraging optical imaging and Optical Coherence Tomography principles, may be supplemented with external ultrasound or ultra-wideband visualization/navigation using an external contact transceiver on the sclera of the eye. Consequently, the apparatus facilitates acute sight-sparing surgeries to be performed on the ocular globe body, key posterior chamber, and lens area structures without touching the cornea. Therefore, the surgical procedure is less likely to trigger an inflammatory healing response, allowing in-situ corneal inflammation to subside before performing a corneal transplant to restore the patient's visual path. By minimizing inflammation and trauma to the prospective implant site, the patient's corneal area will be more receptive to a transplant. In addition, the corneal transplant will be less susceptible to collateral damage or disturbance since other surgeries have been completed a priori.

It should be apparent by the described neurosurgical and ophthalmic applications that the probe serves as a foundational imaging solution to support the development of a new “tool kit” of instruments with integrated visualization of sufficient capability (variable resolution, range, discrimination) to support the confident implementation of procedures that previously relied on non-real-time assessments of targeted areas. In one configuration, the probe is designed and configured to serve as an insert for use in existing hypodermic needles used by surgeons. In other configurations, the probe may be adapted to other surgical instruments.

The probe leverages both single-core and multi-core fibers in Optical Coherence Tomography to support 2D scanning and 3D volumetric reconstruction despite limited space available on the surgical tool, including needles. Contemporary beam scanning techniques (MEMS, piezo actuators, phased arrays, etc.) are comparatively large, with the smallest device outside diameters sized about 1.2-1.5 mm for piezo scanners and larger still for MEMS devices. The probe provides precision imaging and guidance in an extremely small form factor that allows the surgeon to visualize tissue in a forward-looking manner in real-time. The multi-core fiber allows for variable rotation speeds to adapt to various tissue characteristics. For example, extremely sensitive brain tissue that could be inadvertently traumatized by applying higher rotational speeds, can be imaged at a lower rotational speed and still produce real-time, high-resolution imaging of the target tissue.

### BRIEF DESCRIPTION OF THE SEVERAL VIEWS OF THE DRAWINGS

These and other features, aspects, and advantages of various embodiments of the present invention will become better understood regarding the following description, appended claims, and accompanying drawings where:

FIG. 1 is a cross-sectional view of an OCT-enabled precision imaging probe according to an exemplary embodiment of the present invention;

FIG. 2 is a cross-sectional view of an OCT-enabled precision imaging probe according to an alternative embodiment of the present invention;

FIG. 3 is an illustration of the rotational forward-looking scanning configuration of the probe according to one embodiment of the present invention;

FIG. 4 is a schematic view of an exemplary imaging scanning and interrogation pattern generated by the probe according to one embodiment of the present invention;

FIG. 5 is a cross-sectional view showing an exemplary image of a multicore fiber for use in embodiments of the present invention;

FIG. 6 is a cross-sectional side view of one embodiment of the probe;

FIG. 7 is an image of the OCT-enabled precision-guided needle and insert according to an exemplary embodiment of the invention;

FIG. 8 is a flowchart illustrating basic steps of a method for reconstructing an image of tissue interrogated by the probe, according to the inventive subject matter;

FIG. 9 is a flowchart illustrating steps associated with mapping of the spirograph pattern step of FIG. 8 in greater detail;

FIG. 10 is a view of the transform input step of FIG. 8 in greater detail;

FIG. 11 is a view of the interpolate step of FIG. 8 in greater detail;

FIG. 12A is a first low density spirograph interrogation pattern according to the inventive subject matter;

FIG. 12B is a second medium density spirograph pattern according to the inventive subject matter;

FIG. 12C is a third high density spirograph pattern according to the inventive subject matter;

FIG. 13 is a view of a sample tissue target;

FIG. 14A is a view of a low resolution first sorted input associated with FIG. 13 according to the inventive subject matter;

FIG. 14B is a view of a medium resolution second sorted input associated with FIG. 13 according to the inventive subject matter;

FIG. 14C is a view of a high resolution third sorted input associated with FIG. 13 according to the inventive subject matter;

FIG. 15A is a view of low resolution first transformed input associated with FIG. 13 according to the inventive subject matter;

FIG. 15B is a view of a medium resolution second transformed input associated with FIG. 13 according to the inventive subject matter;

FIG. 15C is a view of high resolution third transformed input associated with FIG. 13 according to the inventive subject matter;

FIG. 16A is a view of a first interpolated low-resolution output image associated with FIG. 13 according to the inventive subject matter;

FIG. 16B is a view of a second interpolated medium-resolution output image associated with FIG. 13 according to the inventive subject matter;

FIG. 16C is a view of a third interpolated high-resolution output image associated with FIG. 13 according to the inventive subject matter;

FIG. 17 is an illustration of the use of an embodiment of the inventive subject matter applied to the detection and removal of shrapnel from a subject's eye;

FIG. 18A is a perspective view of an OCT-assisted nerve ablation procedure for spinal nerves.;

FIG. 18B is an amplification of the procedure shown in FIG. 18A;

FIG. 19A is a view of a pre-ablation OCT image; and

FIG. 19B is a view of a post-ablation OCT image.

The accompanying drawings numbered herein are given by way of illustration only and are not intended to be limitative to any extent. Commonly used reference numbers identify the same or equivalent parts of the claimed invention throughout the several Figures.

### OBJECTS

A first object of various embodiments of the present invention is to provide a probe for intra-operative ocular imaging to preserve corneal viability while allowing critical anterior and posterior chamber procedures to be performed safely and effectively.

Another object is to provide a precision image-guided therapeutic needle insert for the neurology field, specifically for precision injection of neurolytics near nerves and delivery of radiofrequency and laser nerve ablation to support, for instance, palliative pain care.

A further object is to provide a precision image-guided tool to allow the precise placement of microelectrodes for neuromodulation and neurostimulation.

An additional object is to provide a precision image-guided tool to simplify and improve the success rate for retrobulbar and epidural injections, among others.

Another object is to provide a precision image-guided needle insert sized to be compatible with any standard surgical insertion needle.

Another object is to provide a probe having a multi-core optical fiber in the scanning mechanism of an OCT-enabled precision image-guided needle to reduce the size, weight, complexity, and manufacturing cost of the probe as compared to the use of a single-core optical fiber.

Another object is to provide a probe having OCT imagery in combination with micro-endoscopic imagery.

Another object of the invention is to provide a nerve tissue ablation apparatus having optical imaging capabilities which allow incremental visual monitoring of tissue to support the accurate placement of a tip of the ablation apparatus relevant to the target tissue site, to assist with verification of treatment effectiveness and to help avoid undesirable lesioning of surrounding tissues, which could be detrimental to the patient.

Other objects of the invention will become apparent through further review of the present disclosure.

### Element List

The following list includes reference numerals associated with elements comprising the inventive subject matter. These elements, and hence, reference numerals, may be found on one or more figures associated with this disclosure. Further detail associated with each of the elements can be found in the detailed description of this disclosure.

Element 10 is a first version of an imaging probe, according to the inventive subject matter.

Element 11 is a surgical introducer needle used to house and introduce components of the imaging probe 10.

Element 12 is an interior wall of the introducer needle 11.

Element 20 is a hypotube insert.

Element 21 is an interior wall of the hypotube insert 20.

Element 22 is a seam joining a hypotube fiber carrier 30 and a second complementary hypotube channel 40.

Element 23 is an annular interior space within the introducer needle 11.

Element 24 indicates the availability of simultaneous bidirectional rotation of hypotube inserts 30, 40 within the annular interior space 23 of the introducer needle 11.

Element 25 represents a center axis of rotation of the hypotube inserts 30, 40 within the annular interior space 23 of the introducer needle 11.

Element 30 is a first hypotube 20 which serves as an optical fiber carrier 30 within the introducer needle 11.

Element 31 represents a center axis of the optical fiber 33 deployed within the optical fiber carrier 30.

Element 32 is an annular lumen within the optical fiber carrier 30.

Element 33 is the optical fiber deployed within the optical fiber carrier 30.

Element 34 indicates bidirectional rotation of the optical fiber 33 within the optical fiber carrier 30.

Element 35 is a distal face of the optical fiber 33.

Element 40 is a second complementary hypotube which can serve as a therapeutic channel and establishes a spacer element adjacent the opposing hypotube 30 within the introducer needle 11.

Element 41 is a therapeutic lumen in the hypotube 40 through which other activities and treatments may take place while using the imaging probe 10.

Element 50 is an alternative imaging insert deployable within the introducer needle 11 and providing alternative rotational scenarios during use.

Element 51 illustrates the available bidirectional rotation of the optical fiber carrier 30.

Element 52 illustrates the available bidirectional rotation of alternative imaging insert 50.

Element 53 is a seam joining therapeutic delivery channel hypotube 40 to the inside wall 55 of the alternative imaging insert 50.

Element 57 is the annular lumen of the second imaging insert 50, which may be used for the additional delivery of therapeutic agents.

Element 60 is one example of a spiral-geometry scanning pattern applied via the imaging probe 10 to create an image of target tissue.

Element 61 is an angle associated with the deflection of an optical beam via the distal fiber tip face 35 which establishes a forward-looking field of view associated with the imaging probe 10 greater than the area of the optical fiber face 35.

Element 62 represents the focal plane associated with the operation of the imaging probe 10, applied to a tissue portion 200.

Element 70 is a multicore fiber that may be deployed within the imaging probe 10 vs a single core optical fiber.

Element 71 is an individual optical fiber core within the multicore fiber 70.

Element 72 is fiber cladding that insulates between each of the individual fiber cores 71.

Element 100 is a diagram of the primary components of an OCT system used in conjunction with the imaging probe 10.

Element 101 is a laser source associated with the OCT module.

Element 102 is a circulator associated with the OCT module.

Element 103 is a fiber optic switch associated with the OCT module.

Element 104 is a multiplexer associated with the OCT module.

Element 105 is a detector associated with the OCT module.

Element 110 is a control assembly associated with the handheld imaging probe 10.

Element 120 is an external female housing sized to movably receive other elements.

Element 130 is a male linear/rotational control member, i.e., a grip.

Element 140 is an internal male mandrel deployed within the female housing 130.

Element 150 is a motor providing in one instance rotation of the probe 10 components.

Element 160 is an optical slip ring provided adjacent to the proximal end of the mandrel 130 to deliver optical signals.

Element 170 is an extended optical and motor controller connector.

Element 200 is a representation of the planar position of biological tissue.

Element 210 is an eye.

Element 211 is a sclera.

Element 212 is a retina.

Element 220 is a spine.

Element 221 is a nerve.

Element 222 is a biological structure.

Now turning to elements associated with the process of developing an image of target tissue according to the inventive subject matters, steps of the visualization method and process follow:

Element 1000 is an image reconstruction algorithm comprising various steps described below.

Element 1001 is a “scan target” step.

Element 1002 is a “sort input” step.

Element 1100 is a “transform input” step.

Element 1101 is a “transform frame” loop step.

Element 1110 is a “transform row” loop step.

Element 1111 is a “check additional transform rows” step.

Element 1120 is a “transform read loop” step.

Element 1121 is a “lookup voxel location” step.

Element 1122 is a “map read to voxel” step.

Element 1123 is a “check additional transform reads” step.

Element 1200 is an “interpolate” step.

Element 1210 is an “interpolate frame loop” step.

Element 1211 is a “display frame” step.

Element 1220 is an “interpolate voxel loop” step.

Element 1221 is a “check for null value” step.

Element 1222 is a “locate nearest read voxel” step.

Element 1223 is a “copy nearest non-null voxel” step.

Element 1224 is a “check for additional voxels” step.

Element 2000 is a “map spirograph pattern” step.

Element 2001 is a “map frame loop” step.

Element 2010 is a “map row loop” step.

Element 2011 is a “check additional map rows” step.

Element 2020 is a “map read loop” step.

Element 2021 is a “calculate voxel location” step.

Element 2022 is a “map voxel location” step.

Element 2023 is a “check additional map reads” step.

Element 3010 is a first low-density spirograph pattern.

Element 3011 is a second medium-density spirograph pattern.

Element 3012 is a third high-density spirograph pattern.

Element 3020 is a sample target to be imaged.

Element 3030 is a first low density sorted input of the sample target 3020.

Element 3031 is a second medium density sorted input of the sample target 3020.

Element 3032 is a third high-density sorted input of the sample target 3020.

Element 3040 is a first low density transformed input of the sample target 3020.

Element 3041 is a second medium density transformed input of the sample target 3020.

Element 3042 is a third high-density transformed input of the sample target 3020.

Element 3050 is a first low density interpolated image of the sample target 3020.

Element 3051 is a second medium density interpolated image of the sample target 3020.

Element 3052 is a third high-density interpolated image of the sample target 3020.

Now, turning to aspects of an OCT-assisted procedure in conjunction with the use of the imaging probe 10 according to the inventive subject matter, other components are listed below:

Element 4000 is an illustration of other elements of an OCT-assisted optometric procedure.

Element 4001 is a debris removal tool used in the procedure.

Element 4002 is a foreign object to be removed by the debris removal tool 4001.

Element 5000 is an illustration of an exemplary approach for a high precision nerve ablation procedure affected using the apparatus according to the inventive subject matter.

Element 5001 is an ablation apparatus.

Element 5002 is an exemplary pre-ablation OCT image.

Element 5003 is an exemplary post-ablation OCT image resulting from the effective use of the imaging probe 10.

Element 5004 represents an ablated region in target tissue shown transecting the nerve tissue 221 while avoiding collateral tissue 222 of noninterest.

### DETAILED DESCRIPTION

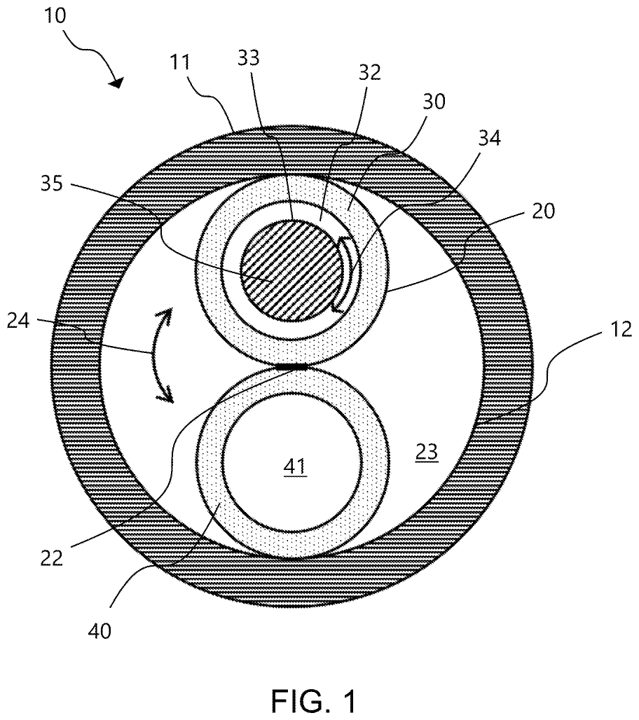

FIGS. 1 through 19B, wherein like parts are designated by like reference numerals throughout, illustrate example embodiments of an OCT-enabled precision image-guided surgical probe according to the inventive subject matter described herein. Although the invention will be described with reference to the example embodiments illustrated in the figures, it should be understood that many alternative forms can embody the inventive subject matter. One of skill in the art will additionally appreciate diverse ways to alter the parameters of the embodiments disclosed, such as the size, shape, or type of elements or materials, in a manner still in keeping with the spirit and scope of the inventive subject matter described herein.

FIG. 1 is an illustration of a cross-sectional view of a first exemplary embodiment of the OCT-enabled precision imaging probe 10. A surgical introducer needle 11 longitudinally encloses a dual parallel hypotube insert 20 having two hypotube working channels: a fiber carrier hypotube channel 30 and a therapeutic hypotube channel 40. Annular space 23 within the probe 10 serves as an additional lumen to support the delivery of fluid therapeutics, including but not limited to, a saline flush, aspiration, contrast agent introduction, and, local injection, among others. In one instance, the parallel hypotube insert 20 preferably comprises two 27-gauge hypotube channels, including the fiber carrier 30 and the therapeutic channel 40, disposed within a surgical introducer needle 11 of 18-gauge size. The fiber carrier 30 and therapeutic channel 40 are joined along their length at seam 22 such that the entire hypotube insert 20 translates linearly and rotationally within the surgical introducer needle 11.

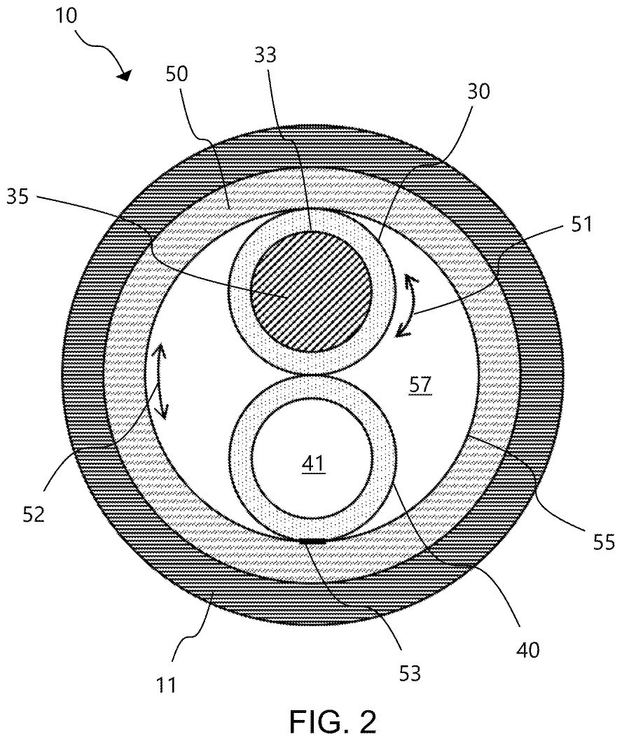

Referring now to FIG. 2, the cross-section for an alternative embodiment of the inventive subject matter is shown. In this configuration, imaging probe 10 comprises a surgical introducer needle 11 for receiving an alternative imaging insert 50. The alternative imaging insert 50 is linearly and rotationally translatable within the interior of the surgical introducer needle 11. The alternative imaging insert 50 includes a fiber carrier 30 and therapeutic channel 40. The therapeutic channel 40 is joined via a seam 53 along its length to an interior wall 55 of the alternative imaging insert 50 such that as the alternative imaging insert 50 rotates, the therapeutic channel 40 likewise translates in a circular pattern about the center axis of the alternative imaging insert 50.

In contrast to the first embodiment, the fiber carrier 30 is not joined to the interior wall 55 of the alternative imaging insert 50. Thus, the fiber carrier 30 may both linearly translate and undergo second fiber rotation 51 independently of the interior wall 55 of the alternative imaging insert 50. The fiber carrier 30 includes an optical fiber 33 bonded to the interior of the fiber carrier 30 such that as the fiber carrier 30 rotates, the optical fiber 33 is likewise caused to rotate. The alternative imaging insert 50 and therapeutic channel 40 having lumen 41 will rotate in either a clockwise or a counterclockwise direction as shown by the second insert rotation 52. The fiber carrier 30 rotates independently in either a clockwise or a counterclockwise direction as shown by the second fiber rotation 51.

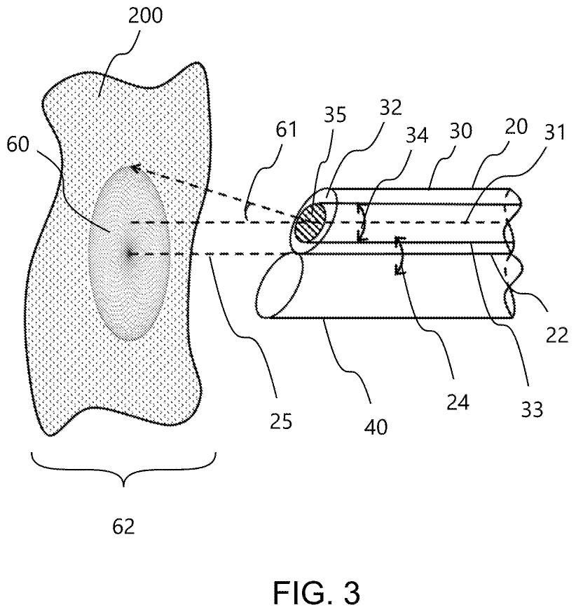

Now, referring to FIG. 3, in greater detail, the structure and function of the hypotube insert 20 of the imaging probe 10 are described. The fiber carrier 30 comprises a lumen 32 through which an optical fiber 33 is longitudinally disposed in a manner to allow both linear translation and rotation 34 of the optical fiber 33, through which imaging is achieved. The fiber carrier 30 provides scanned OCT with voxel resolution on the order of 20 μm (Z-propagation axis)×50 μm (X)×50 μm (Y) in a forward-looking mode with a full angle of deflection on the order of 60 to 70 degrees from the center axis 31 of the optical fiber 33. A scanning pattern is established via rotation 24 of the hypotube insert 20 and fiber rotation 34 of the optical fiber 33 within the fiber lumen 32. A plurality of rotational speeds may be applied to the hypotube insert 20 and fiber carrier 30 in either clockwise or counterclockwise directions. One or more algorithms created to acquire, interpret, and analyze the resulting optical signals are implemented via a computer processing unit and display system to generate a meaningful real-time or near real-time image for presentation to a caretaker, e.g., surgeon, physician, etc., during a procedure.

The imaging probe 10 is configurable to provide improved precision neurosurgery and to support, among other procedures, precision RF/laser ablation of nerves for palliative pain relief from metastases in end-stage cancer patients. In addition, the imaging probe 10 facilitates precision injection of nerve modulating agents, e.g., anesthesia, the precise placement of microelectrodes for neuromodulation and neurostimulation, and the introduction of contrast agents in support of OCT and micro-endoscopic imaging. In one aspect, the hypotube insert 20 is preferably deployed via a standard 18-gauge Touhy-type hypodermic surgical introducer needle 11. However, the hypotube insert 20 is scalable for use with needles larger and smaller than 18-gauge in size.

The imaging probe 10 delivers optical components via the hypotube insert 20 to support Optical Coherence Tomography (OCT) in a compact form factor for insertion in a standard hypodermic surgical introducer needle 11. The imaging probe 10 provides imaging via a forward-looking scanning mechanism to provide an adaptable resolution that allows a surgeon to view different tissue features depending on the type of surgery. In the exemplary embodiment, the forward-looking resolution of the imaging probe 10 is approximately ten to fifteen microns laterally across approximately one to two millimeters of penetration depth in scattering tissue T. Penetration depth may extend to more than one centimeter in clear tissue, CSF, the vitreous and aqueous humor, and other similar tissue environments. The compact OCT and micro-endoscopic imaging capability delivered via the imaging probe 10 is capable of distinguishing both gross tissue form as well as single distinct cell layers to enhance a surgeon's awareness of the tissue type adjacent and forward of the tip of the imaging probe 10 to increase the probability of treating the desired tissue type and area. The scanning method according to the inventive subject matter allows the surgeon to visualize a larger area of the target tissue 200 to provide a more comprehensive understanding of the surrounding tissue while still enabling the surgeon to focus on specific tissue sites.

The ability of the imaging probe 10 to support highly resolute visualization of target tissue 200 during surgical procedures creates several benefits. A first benefit is a reduction of the extent of collateral damage around a nerve during and following therapeutic ablation or injection.

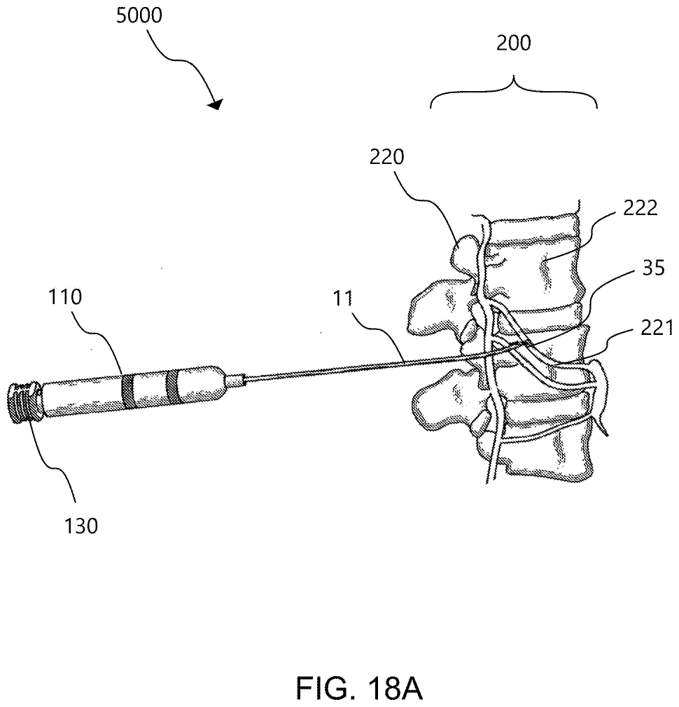

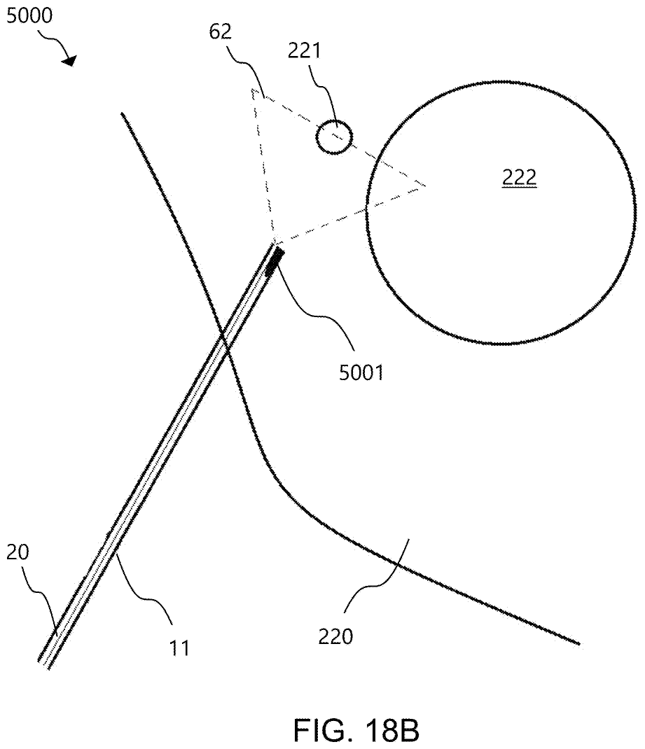

Current fluoroscopy guidance allows a physician to navigate to within a few millimeters of a nerve. In the context of neurosurgery or anesthesia, the accuracy of a few millimeters is considered coarse placement. Additionally, guidance under fluoroscopy does not allow the surgeon to view target tissue 200 during a procedure. Instead, the practiced surgeon must rely on knowledge and expertise developed over many years of trial-and-error practice. Consequently, the subsequent therapy must anticipate this potential inaccuracy and plan for sufficient treatment of an excessive target volume that hopefully includes the targeted tissue 200, such as a nerve 221 (see FIG. 18A, 18B, 19A, and 19B).

For example, radiofrequency heating must coagulate a large tissue volume with major and minor axes of several millimeters since a nerve can be located anywhere within a target zone. At times, where nerves may uniquely traverse a treatment zone for specific individuals, the surgeon will only know if the treatment was successful after the fact when a patient can respond with his or her own assessment. During the actual procedure, the surgeon is unable to view whether the tip of the surgical introducer needle 11 and various therapeutic implements are adjacent and in sufficiently proximity to the target tissue 200. Fluoroscopy-guided treatments assist the surgeon but cannot be relied on confidently, particularly by less seasoned medical practitioners.

Similarly, for the injection of a neurolytic, the injected fluid volume must be large enough to incapacitate a volume of tissue encompassing the potential error in locating the nerve. One injectable, absolute alcohol is highly labile/mobile and tends to diffuse through the tissue, damaging tissues through which it diffuses until diluted down below a non-damaging threshold. Often these treatment methods can create large zones of necrosis that cause as much pain as is being ameliorated at the metastasis site by the destruction of a nerve. Precise targeting afforded by the imaging probe 10 according to the inventive subject matter described herein minimizes the need to treat large volumes in the target area, thereby mitigating the negative side effects with larger treatment volumes.

A second benefit associated with the use of the imaging probe 10 is a reduction of patient re-admit rates. It is often the case that even where overtreatment occurs, as described above, a target nerve is only temporarily incapacitated at the time of surgery and subsequently recovers. Consequently, the patient's pain returns, and the patient is likely to be re-admitted to the medical facility. Re-admits are inconvenient and unfortunate for the patient, not well received by insurance companies, and in most cases, significantly increase expenses associated with the overall treatment. The present invention reduces the re-admit rate by offering more precision in the initial surgery, providing evidence-based support via direct imaging of the results of treatment of target tissue, and ensuring improved nerve incapacitation on a first visit.

The imaging probe 10 comprises a precision hypotube insert 20 scalable to be compatible with a plurality of hypodermic needle sizes. In one instance, the provision of a hypotube insert 20 for deployment via a standard hypodermic needle allows the technology benefits to be available to all physicians performing procedures regardless of their personal needle preferences, i.e., they can keep using their preferred needles and sizes. The components of the imaging probe 10, including the hypotube insert 20, are constructed of biocompatible and sterilizable materials. The fiber carrier 30 used of the hypotube insert of the imaging probe 10 is preferably glass with a polyimide coating that is receptive to sterilization for re-use and known to survive Ethylene Oxide gas sterilization. However, in other embodiments, the fiber carrier 30 may be configured only for single-use and disposed of rather than sterilized. The surgical introducer needle 11 may be standard stainless steel hypodermic needle tubing. Other materials used in imaging probe 10 include sapphire, adhesives, and plastics, such as neoprene and polyurethane. The imaging probe 10 is comprised of materials accepted as being biocompatible and nontoxic, suitable for use in neural surgery and reconstructive surgery around the eye including vitreous replacements, saline, insufflation gases, silicone oils, etc.

Referring still to FIG. 3, the means by which the probe 10 delivers forward-looking precision imagery via OCT and other imaging modalities, such as micro-endoscopy, is described. A dual rotational scanning methodology is shown in FIG. 1, FIG. 2 and FIG. 3 consistent with hypotube insert 20 configuration for the two embodiments. A similar dual rotational scanning method may be applied using the alternative imaging insert 50 of FIG. 2.

As shown in FIG. 3, the hypotube insert 20 may rotate in either a clockwise or a counterclockwise direction about its insert center axis 25, as indicated by the insert rotation 24, while deployed within the surgical introducer needle 11. The optical fiber 33 deployed within the lumen 32 of the imaging fiber carrier 30 will likewise rotate in either a clockwise or counterclockwise direction about its fiber center axis 31 as indicated by fiber rotation 34. The optical fiber 33 includes a distal fiber tip 35, which is polished such that the laser light transmitted through the optical fiber 33 exits from the fiber tip 35 of the optical fiber 33 at an angle 61. The angle 61 may be varied by modifying the fiber tip 35 of the optical fiber 33. Variations of angle 61 will change the scanning extent and scanning pattern 60 of the hypotube insert 20 on the targeted tissue 200, along with the focal plane 62.

The fiber tip 35 may include other optical components for further refinement and adjustment of optical signal transmission and reception. For example, fiber tip 35 may include an optical sapphire lens. Alternatively, fiber tip 35 may include other lens material with a high refractive index such as undoped yttrium aluminum garnet (YAG) which will provide sufficient hardness, includes a high refractive index, is more easily polished, and is not birefringent and thus maintains polarization of the light. Various lens configurations allow probe 10 to produce a wider field of view. In one instance, probe 10 is adept at targeting large structures, e.g., 1 mm in diameter, at 2 to 3 millimeters distance away from the target structure. In conjunction with the spirographic scanning pattern afforded by the inventive subject matter, a very dense point cloud may be generated to support volumetric microscopy, e.g., for pathology.

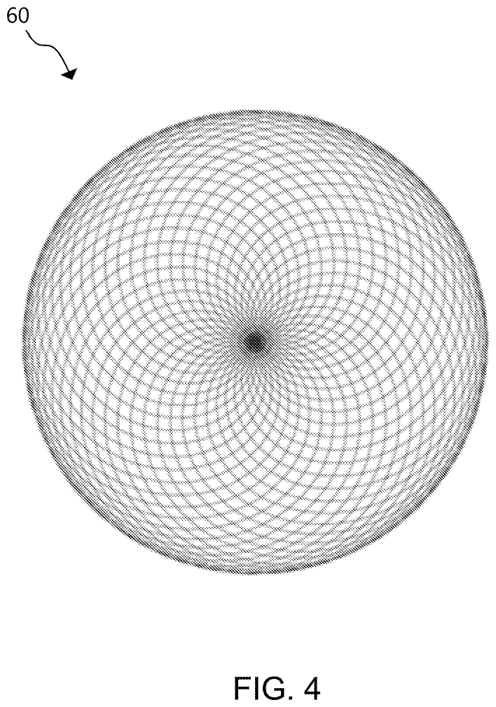

Referring now to FIG. 4, a schematic of an exemplary scanning pattern 60 of the imaging probe 10 is shown. A combination of off-axis and on-axis rotation of the primary elements of the hypotube insert 20 generates a “Spirograph”-like scanning pattern 60. In use, the combination of rotation of the optical fiber 33 and/or the fiber carrier 30 about a fiber center axis 31 and rotation of the entire hypotube insert 20 about the insert center axis 25 causes a transmitted OCT beam to precess analogous to a “Spirograph” scanning pattern 60 like that shown in FIG. 3 and FIG. 4. The Spirograph scanning pattern 60 is directed in a forward-looking manner, supporting the acquisition of highly resolute tissue information and imagery while expanding the areal extent of the acquired image beyond the areal extent of the fiber tip 35.

The scanning pattern 60 may be varied by varying the rotational speed of the fiber carrier 30 and hypotube insert 20, and, by changing the lens geometry at the fiber tip 35, along with changing the sampling frequency. Thus, a sampling density (point cloud density) may be adapted to suit specific tissue requirements or procedural requirements. For example, a slow but dense scan pattern may be used to confidently locate an object/target of interest, identify the target, and accurately triangulate its position prior to approach. Once a target is located, a faster less-dense scan pattern can be implemented to monitor the target during treatment. For example, the less dense scan pattern may be used to assist with imaging to support accurate titration of the duration or intensity of therapy. For example, probe 10 can assist in managing the duration and power applied to a nerve when ablating.

By employing OCT in conjunction with the forward-looking scanning methodology associated with multiple embodiments of the invention, the imaging probe 10 can provide a surgeon with a two-dimensional and/or three-dimensional visualization of the target tissue 200 in real-time or near real-time. Consequently, the imaging probe 10 may be used in a stereoscopic manner with advanced displays, including virtual reality and augmented reality displays, for example, the OCULUS RIFT, the MICROSOFT HOLOLENS, and other wearable or heads-up type displays.

Referring once again to the embodiments illustrated in FIG. 1 and FIG. 2, common elements are described in greater detail. A therapeutic channel 40 supports the entry and passage of other tools through the therapeutic lumen 41 to support the performance of various surgical procedures. In many cases, these procedures are directed to neural and optical requirements but may be extended to other scenarios encountered by a surgeon where forward-looking, high-resolution imagery can prove useful.

For example, a surgeon may use therapeutic lumen 41 to place an RF electrode or optical fiber (laser delivery) in close proximity to target tissue 200 to perform ablation or coagulation of one or more nerves. The lumen 32 may also be used to heat the therapeutic channel 40 comprising a stainless steel hypotube to ensure that an RF or laser ablation process occurs in the desired temperature range.

During ablative or coagulative therapy, the treatment will typically modify the optical properties of a target tissue 200. The imaging probe 10 allows the surgeon to continuously monitor the target tissue 200 during treatment to both enhance the precision of the treatment, confirm that the treatment has been successful, and avoid overtreatment. In another aspect, the therapeutic lumen 41 of the therapeutic channel 40 can be used to precisely place extremely small electrodes adjacent to target tissue 200 to support neuromodulation. Still further, the therapeutic channel 40 may be used to deploy an optical fiber bundle to support micro-endoscopy as a supplemental imaging solution.

Referring again to FIG. 1, the imaging probe 10 includes a “bat-wing” shaped area formed between the introducer needle interior wall 12 of the surgical introducer needle 11 and the exterior of the fiber carrier 30 and the therapeutic channel 40, creating an additional annular space 23. The annular space 23 provides an alternative pathway that a surgeon may use for flushing saline fluids to a target site, aspiration, precise injection of neurolytics/paralytics, injection of contrast agent and/or continuous flow of cooling fluids during treatment of target tissue 200. There are several synergies with ophthalmic surgical techniques where the availability of the therapeutic lumen 41 in conjunction with the therapeutic channel 40 is useful.

In another aspect, related to ophthalmic surgical procedures, the imaging probe 10 may use 2.94-micron pulsed erbium: YAG laser transmitted through a fiber optic as a vitrectomy system, allowing the imaging probe 10 to “burrow” through the vitreous humor minimizing barotrauma which could trigger further inflammation of tissues. In combat injury situations with multiple lacerations, there is a need to avoid excess pressure near or on ophthalmic surgical repair sites, as these are weaker than the surrounding intact tissue. Excess pressure may risk the failure of sutures or the nucleation of an inflammatory signaling response in the target tissue 200. As surgery in the eye is commonly performed with manual support and eye tissues are delicate, the imaging probe 10 allows the surgeon to use tools that are smaller and lighter and deliver critical functionality such as phacoemulsification, pneumatic vitrectomy, and aspiration et al.

As previously described, the hypotube insert 20 (FIG. 1) and alternative imaging insert 50 (FIG. 2) both employ dual rotational scanning to support forward-looking imagery during surgical procedures. Dual rotation requires two means of rotation running at two rotation speeds to produce a well-characterized point cloud. To support video-rate imaging, one rotational mechanism may support a rotation speed of approximately 1000 rpm. Hence, in an alternative aspect, other embodiments in accordance with the invention can benefit from means to reduce the rotational speed of the scanning elements.

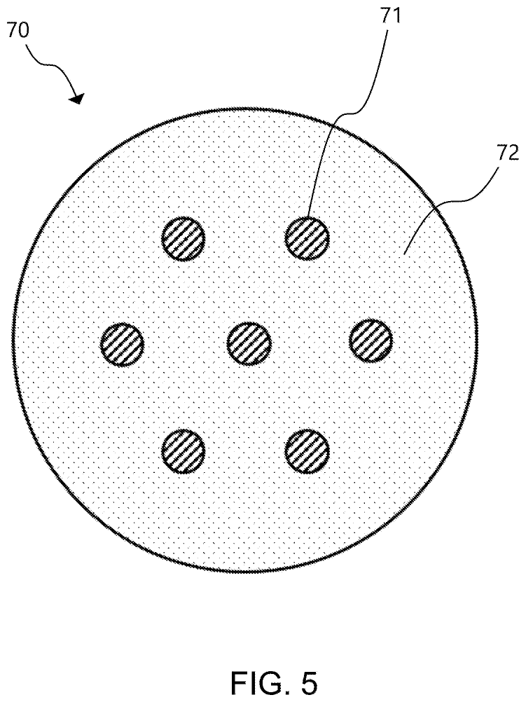

Referring now to FIG. 5, one approach used for reducing required rotational speeds is via the use of a multicore fiber 70. The cross-section of a multicore fiber 70 having seven fiber cores 71 is shown. In alternative embodiments, the multicore fiber 70 may be used for OCT rather than a single core OCT optical fiber 33. The multicore fiber 70 provides a multiplexing advantage in data acquisition. The multicore fiber 70 is back-illuminated and end-face imaged using standard telecom techniques. In one configuration, each fiber core 71 is approximately 10 microns in diameter and fiber cladding 72 has an approximate overall diameter of 125 microns. Although shown herein as having seven fiber cores 71, designs with 19 and 37 fiber cores on hexagonal stacking patterns are available and may be substituted and used in the imaging probe 10. A separate fiber adapter control assembly (110 couples each multicore fiber 70 used within the imaging probe 10 so that the system may separately address each fiber core 71 to acquire optical signals to support more resolute and higher density 2D and 3D images.

Substitution of a multicore fiber 70 can allow the physician to reduce the rotational speed by a factor equivalent to the number of fiber cores 71 in the multicore fiber 70, for example, 7×, 19×, or 37×. An ultimate reduction in the rotation speed of 19× and 37× simplifies the mechanical requirements of the control assembly 110 and rotational drive mechanisms. For example, a multicore fiber 70 may be used in a manner that reduces the need for a second rotational means. Instead, the imaging probe 10 can use a single rotational means transmitting rotation to both rotating portions through a meshed geared arrangement. Additionally, a more slowly rotating multicore fiber 70 decreases vibration which might be caused by higher speed drives, thereby reducing the likelihood of binding, or galling of the various components of the insert probe 10. In an ophthalmic procedure, galling could cause fine debris to be shed intra-ocularly which could be catastrophic in a procedure. The inclusion of multicore fiber 70 also allows the hypotube insert 20 to scale to smaller sizes for use in small needles and other devices with restricted working channels, including endoscopes, needle biopsy devices, and robotic surgery instruments.

As the number of fiber cores 71 are increased, the size of the multicore fiber 70 may also increase. The imaging probe 10 provides flexible and adaptable configuration such that appropriate tradeoffs can be made to optimize the configuration to each specific use case. For example, a photonic crystal multicore fiber 70 having thirty-seven fiber cores 71 has an overall diameter on the order of 250 microns, compatible with insertion in a small gauge hypotube and capable of providing volumetric imaging, structural information, and some molecular information using the dual rotational technique in accordance with the inventive subject matter described herein, while still allowing deployment of the imaging probe 10 in a very small profile device. Such a configuration will be advantageous in neurovascular surgery (strokes, local injection of thrombolysis agents or laser thrombolysis), cardiovascular surgery using catheters (laser treatment of heavily calcified chronic total occlusions where the consequences of perforation are too severe to contemplate using an unguided device), and, robotic surgery with small incision sites where collision avoidance and target registration are critical to outcomes, for example, in nerve-sparing surgery around the prostate or ureter/urethra.

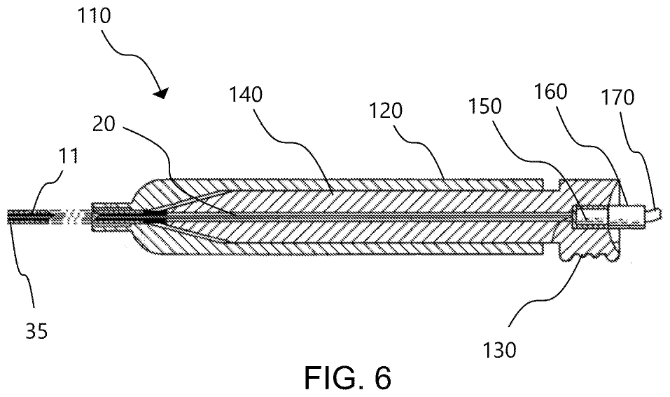

Referring now to FIG. 6, a side cross-sectional view of one version of a control assembly 110 incorporating the probe 10 is shown. The control assembly 110 includes the introducer needle 11. The control assembly 110 includes a female housing 120 and a male linear/rotational control member 130. The male control member 130 is connected to a mandrel 140. The mandrel 140 is slidably and rotatably received within the female housing 120. Probe 10 is connected to control member 130 of the control assembly 110. The control member 130 is further electrically and optically connected to an external system monitoring and control unit (not shown herein).

One or more motors 150 are incorporated in a proximal end of the control assembly 110 to provide means for rotation to the components of the probe 10. In one configuration, only one motor 150 is used to provide means for rotation, and gearing is used to drive the other rotational component. In another configuration, only one motor 150 is used to provide means for rotation of one rotational component, and manual manipulation of the mandrel 140 provides rotation of a second rotational component. An optical slip ring 160 is provided adjacent to the proximal end of the mandrel 130 to deliver optical signals via an extended optical and motor control connector 170 to a control and display system.

In further detail, the control assembly 110 is comprised of a female housing 120 and a linear/rotational control member 130. The control member 130 is attached to an elongate, cylindrical mandrel 140. The mandrel 140 is sized to slidably and rotatably mate within the female housing 120. The mandrel 140 is affixed to the control member 130 such that the mandrel 140 may be reciprocated or rotated within the female housing 120 by manipulation of the control member 130. Probe 10 extends through the length of the mandrel 140. An optical and electrical connector 170 connects with the optical slip ring 160 and rotational motors 150 in the control member 130. An infusion port may be disposed on the introducer needle 11 for delivery of various fluidic media to a target treatment site.

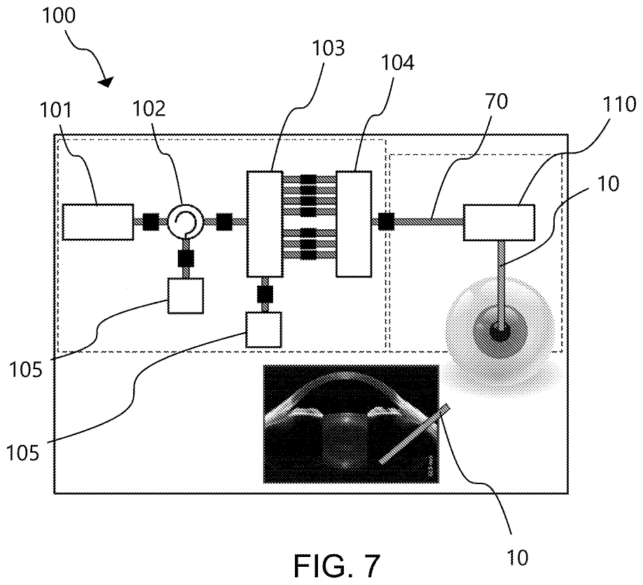

Referring now to FIG. 7, elements of the fiber-multiplexing schema and OCT system 100 according to an embodiment of the invention are shown. In this embodiment, the OCT system 100 uses a 7-core fan-out multiplexer 104 to support a seven-core multicore fiber 70. In other aspects, a multicore fiber 70 having more than seven fiber cores 71 may be used with appropriate multiplexer designs. In the present OCT system 100, 7-core multicore fiber 70 is connectorized with appropriate accuracy for integration with the multiplexer 104. Fast fiber-optic switches 103 for telecom wavelengths may be used to transmit, collect, and integrate optical signals recovered via the imaging probe 10. In another aspect, multiple 1×2 fiber optic switches 103 may be used in place of a single 1×8 fiber optic switch 103 to increase the data acquisition rate. An OCT engine, such as that available from NTT ADVANCED TECHNOLOGY, may be used as a laser source 101 for the OCT imaging. Additionally, a fiber-optic slip ring 90 is utilized to enable continuous rotation of the optical fiber 33. The remainder of the fiber multiplexing environment and OCT system 100 may be comprised of common-path interferometer components, including a circulator 102 and detectors 105.

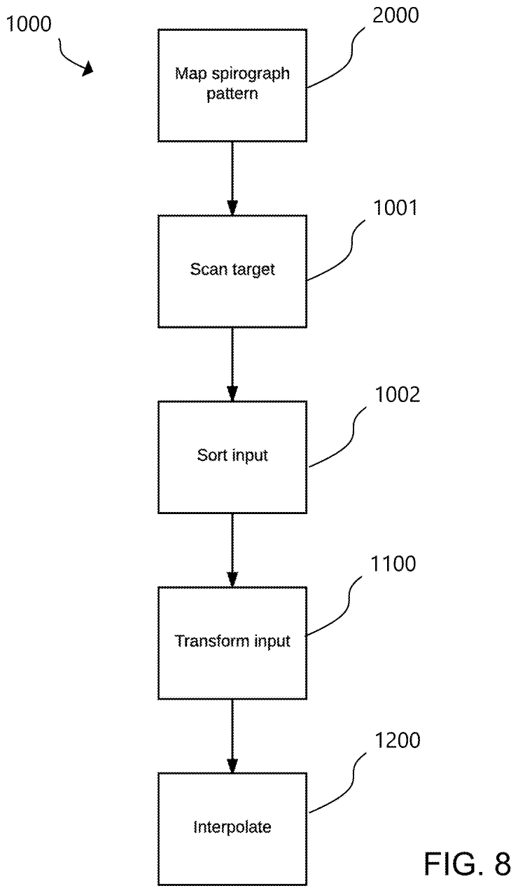

Referring now to FIG. 8, a flowchart describing a method for production of an imaged according to the inventive subject matter For any imaging modality according to the inventive subject matter described herein, a reconstruction algorithm executable on a computer processor is required to render a human-readable image. The method 1000 first includes mapping a spirograph pattern 2000 across target tissue 200 where, important parameters regarding the trace pattern are identified and utilized to determine the pattern shape. These parameters include, but are not limited to, the imaging framerate, the number of voxels scanned per frame, the number of insert rotations 24 of the hypotube insert 20 per frame, the number of fiber rotations 34 of the optical fiber 33 per frame, the number of voxels scanned per insert rotation 24 of the hypotube insert 20, and the location of the first voxel scanned in each frame. With these parameters, a computer processor maps each voxel as it is scanned by the detectors 105 to a specific location on the scanning pattern 60.

Subsequently, probe 10 scans the target 1001 to create a complete pattern 60 corresponding to a single image frame of a video. The signals collected from scanning of the target 1001 are transmitted to the detectors 105 as a 1-dimensional digital input stream of voxel intensities. Next, the 1-dimensional input stream is sorted 1002 , is into a plurality of 2-dimensional arrays, with each array corresponding to a single video frame. In one embodiment, the 2-dimensional arrays are sorted so that each row corresponds to all measured voxel intensities recorded during a single fiber rotation 34 of the optical fiber 33.

Once the input is sorted, it is then transformed 1100 wherein each voxel from the sorted input is mapped to its proper 2-dimensional location on a final image. Many scanning patterns 60 may space voxels unevenly around the image space. Therefore, in one version, the method interpolates 1200 missing voxel intensities. Missing voxel intensities are interpolated based on the measured intensities of neighboring voxels. One or more interpolation algorithms may be used to provide values for missing voxel intensities. For example, the method according to the present invention will construct new data points to fill voids based on other known data points within range of a discrete set of surrounding data points.

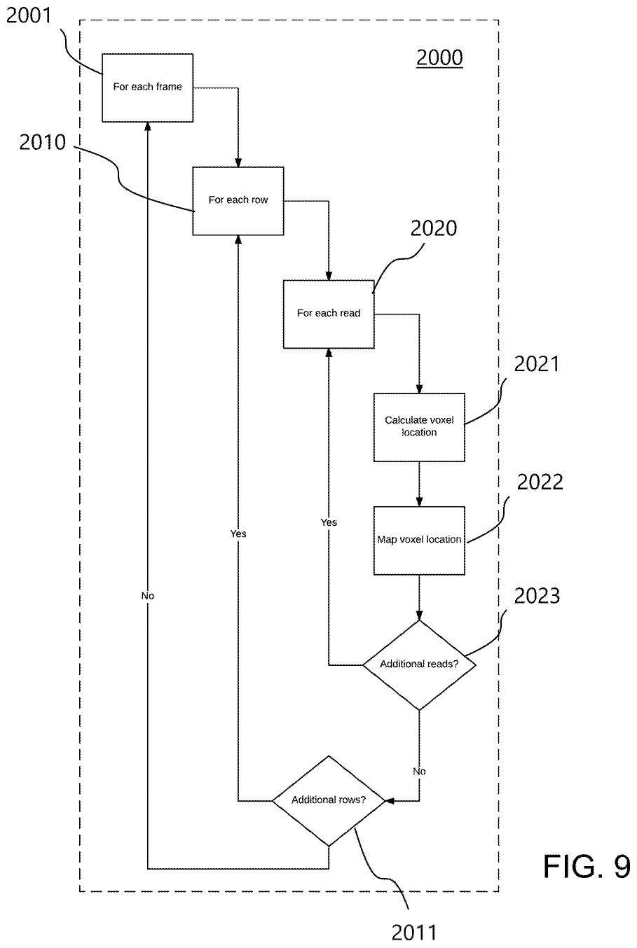

Referring now to FIG. 9, in one configuration, the method for mapping the initial spirograph pattern 2000 is described in greater detail. When mapping the spirograph pattern 2000, the process comprises a series of nested loops. A first map frame loop 2001 iterates across every unique frame index, “n”, in the spirograph pattern, with each frame corresponding to one insert rotation 24 of the hypotube insert 20. A map row loop 2010 iterates across every row index, “j”, in a single frame, corresponding to one fiber rotation 34 of the optical fiber 33. A map read loop 2020 iterates across every distinct input index, “k” recorded within a single row. In calculating a voxel location 2021, the voxel location is calculated for each measurement according to the function (x,y,z)=Fn(j,k), where (x,y,z) is an ordered triplet describing the voxel location, j and k are the row and column indices s for the sorted input, n is the frame number, and Fn is a pattern function describing the spirograph pattern for frame n. In mapping the voxel location 2022, the ordered triplet (x,y,z) is recorded to a mapping table M according to the function Mj,k,n=(x,y,z). The process continues by checking check additional map reads 2023, where the map read loop 2020 is repeated across all inputs within a single row. If additional reads produce data, then additional map rows are checked 2011, with the map row loop 2010 repeating across all rows within a single frame. In a preferred embodiment, the spirograph parameters are set such that the scanning pattern 60 is consistent across all frames n, causing the map frame loop 2001 to require only one iteration. However, the process will repeat as necessary until all voxels relevant to an image are collected.

Referring again to FIG. 8, in scanning the target 1001, the detector 105 collects a 1-dimensional stream of discrete voxel intensities I(t). In sorting the input stream 1002, the 1-dimensional stream of discrete voxel intensities I(t) is mapped to a sorted array I′(j,k,n), where j and k are row and column indexes within a single frame n.

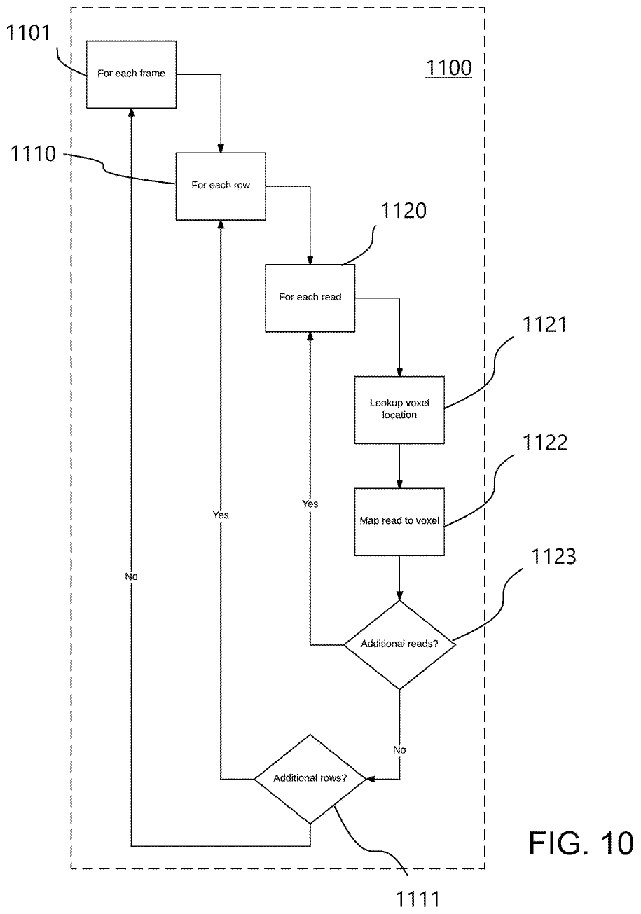

Referring now to FIG. 10, transforming the input stream 1100 is described in greater detail. Analogous to the mapping the spirograph pattern 2000, transforming the input 1100 comprises an iterative process with several nested loops. The transform frame loop 1101 iterates across every frame n. The transform row loop 1110 iterates across every row j within a single frame. The transform read loop 1120 iterates across every distinct input index k within a single row. In lookup voxel location step 1121, the ordered triplet describing a voxel location corresponding to a particular voxel intensity I′(j,k,n) is determined by searching the lookup table Mj,k,n for the corresponding ordered triplet (x,y,z). In map read to voxel step 1122, the voxel intensity is mapped to an image-space array of voxel intensities V according to the function Vx,y,z=I′(j,k,n). In check additional transform reads step 1123, the transform read loop 1120 is repeated across all reads in a single row. In check additional transform rows step 1111, the transform row loop 1110 is repeated across all rows in a single frame.

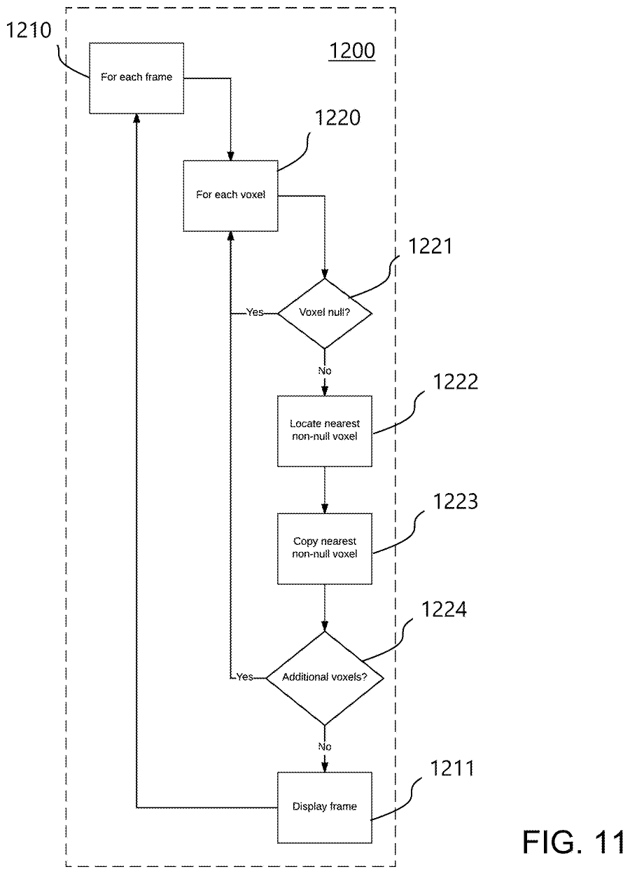

Referring now to FIG. 11, an embodiment of the interpolate step 1200 from FIG. 8, utilizing a nearest-neighbor interpolation algorithm, is described in greater detail. A nearest neighbor interpolation algorithm is one of many embodiments. Other interpolation techniques may be used in the reconstruction of the images and are applicable to the image reconstruction algorithm 1000. These interpolation algorithms may include linear, cubic, multivariable, and gaussian algorithms.

The image-space array of voxel intensities V generated by transforming the input 1100 will typically contain a number of null values within the array. Interpolation 1200 fills in the null values with approximations based on the other recorded values within the array. The interpolate frame loop 1210 iterates across all frames n. The interpolate voxel loop 1220 iterates across all voxels (x,y,z) within a single frame n. Each iteration will check for null values 1221, and each voxel intensity Vx,y,z is checked to determine if the voxel is a null value. If the voxel is non-null, then the voxel intensity has already been measured directly and no interpolation is required. The original value is copied to an array of interpolated voxel intensities V′x,y,z.

If the voxel is a null value, the nearest non-null voxel is determined 1222. For a given ordered triplet (x1,y1,z1), the nearest non-null voxel Vx2,y2,z2 is the voxel that minimizes the distance d=((x1−x2)2+(y1−y2)2+(z1−z2)2) 1/2. The value of the nearest non-null voxel is copied to 1223 to the array of interpolated voxel intensities V′ according to the equation V′xl,y1,z1=Vx2,y2,z2. The interpolate voxel loop 1220 is iterated across all voxels to check for additional voxels 1224. When non additional voxels are located, the the array of interpolated voxel intensities V′ is translated into a human-readable image utilizing a desired format 1211 and the process 1200 is then repeated to generate the next frame.

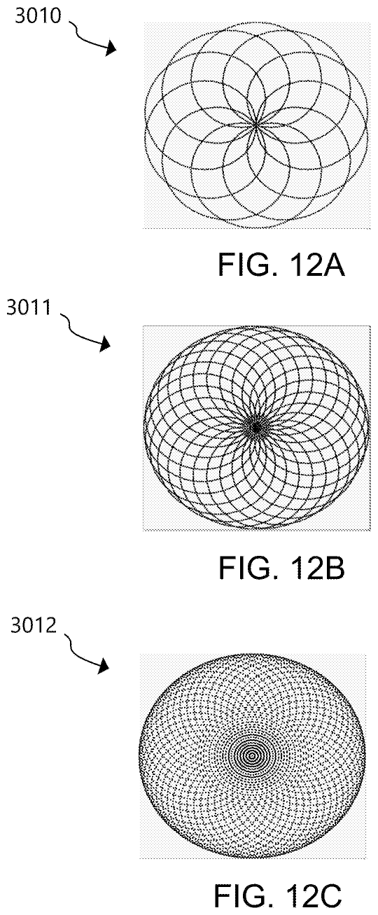

Now with reference to FIGS. 12-16, exemplary illustrations of various stages associated with the spirographic scanning methodology according to the inventive subject matter described herein are shown. These illustrations include example renderings of a target image at various stages in the method for reconstructing an enface image. The examples provided describe a 2-dimensional enface reconstruction with a spirograph scanning pattern 60 that does not vary by frame. In other words, the various pattern functions Fn described do not vary with respect to n, and z has a maximum index of 1. The imaging probe collects sufficient 3-dimensional data and information to extend these examples to 3-dimensional volumetric scans, and spirograph configurations with frame-variable scanning patterns 60.

Referring now to FIGS. 12A-12C, three sample spirograph scanning patterns are shown. The sample spirograph scanning patterns are visual representations of example scanning pattern functions Fn. The first spirograph pattern 3010 represents an example low-resolution scanning pattern with 2000 voxels measured per frame at a ratio of 10 fiber rotations 34 per frame. The second spirograph pattern 3011 represents an example medium-resolution scanning pattern with 4000 voxels measured per frame at a ratio of 25 fiber rotations 34 per frame. The third spirograph pattern 3012 represents an example high-resolution scanning pattern with 6000 voxels measured per frame at a ratio of 50 fiber rotations 34 per frame.

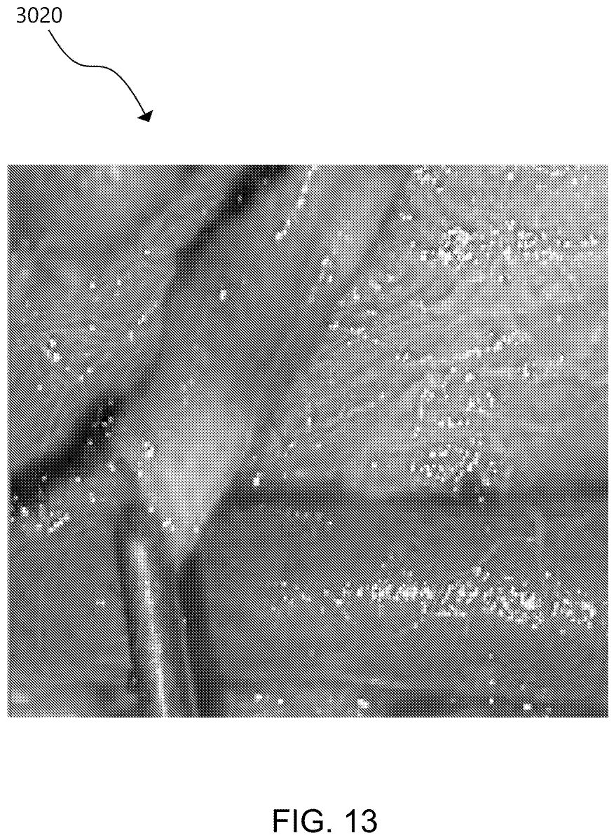

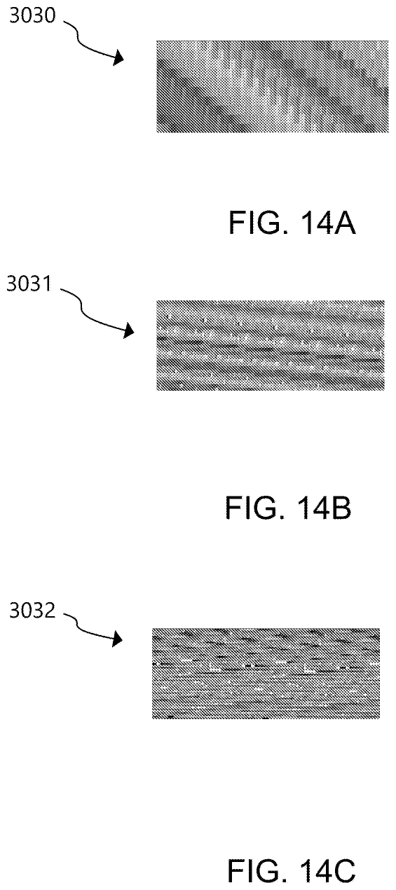

Referring to FIG. 13, an image of a sample target 3020 is shown. In the following FIGS. 14-16, the sample target 3020 is shown at various stages of transformation associated with a scan in a single frame by the OCT system 100 and processed according to the method of the image reconstruction process 1000.

Referring now to FIGS. 14A-14C, example sorted inputs based on a scan of the sample target 3020 are shown. The example sorted inputs are visual representations of the sample target 3020 scanned as a single frame and rendered as a sorted array, I′, according to sort input step 1002. The first sorted input 3030 represents a sorted array collected by the OCT system 100 according to the first spirograph pattern 3010. The second sorted input 3031 represents a sorted array collected by the OCT system 100 according to the second spirograph pattern 3011. The third sorted input 3032 represents a sorted array collected by the OCT system 100 according to the third spirograph pattern 3012.

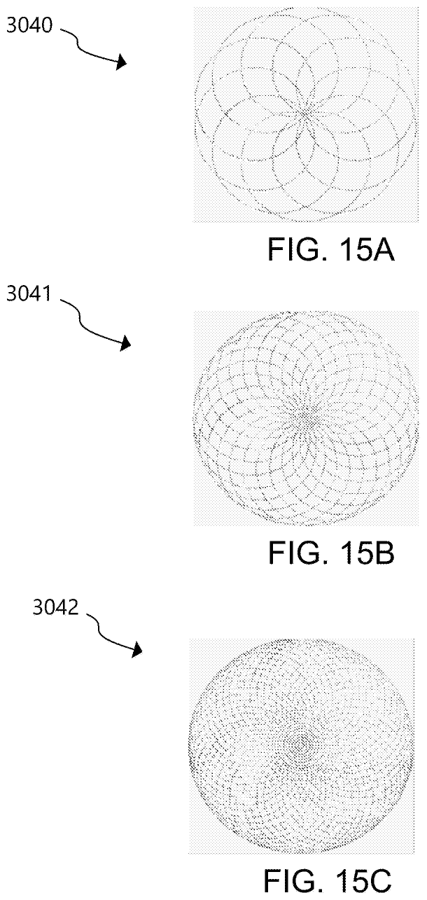

Referring now to FIGS. 15A-15C, example transformed inputs are shown. The example transformed inputs are visual representations of the sample target 3020 rendered as arrays of voxel intensities V according to the transform input step 1100. For display purposes, null values are rendered as white space. The first transformed input 3040 represents an array of voxel intensities created by transforming the first sorted input 3030 according to the scanning pattern represented by the first spirograph pattern 3010. The second transformed input 3041 represents an array of voxel intensities created by transforming the second sorted input 3031 according to the scanning pattern represented by the second spirograph pattern 3011. The third transformed input 3042 represents an array of voxel intensities created by transforming the third sorted input 3032 according to the scanning pattern represented by the third spirograph pattern 3012.

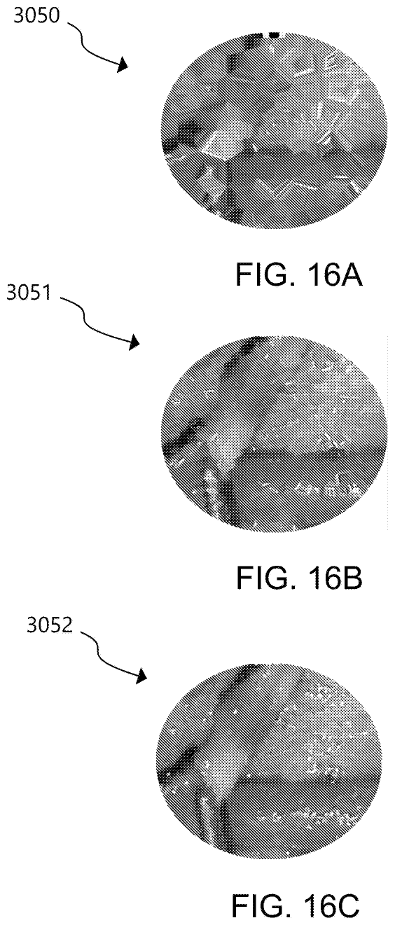

Referring now to FIGS. 16A-16C, example interpolated images at differing resolutions are shown. The example interpolated images are reconstructions of the sample target 3020 as arrays of interpolated voxel intensities V′ according to an embodiment of the interpolate step 1200 utilizing a nearest neighbor interpolation algorithm. The first interpolated image 3050 is an interpolated reconstruction of the first transformed input 3040. The second interpolated image 3051 is an interpolated reconstruction of the second transformed input 3041. The third interpolated image 3052 is an interpolated reconstruction of the third transformed input 3042.

The example illustrated in FIGS. 12-16 describe the reconstruction of a 2-dimensional enface image. For basic imaging applications, this image provides a perspective akin to what a human eye could detect with sufficient magnification. For depth-penetrating imaging modalities such as OCT, this view provides a view at a specified depth from the end of the imaging probe 10. According to the inventive subject matter described herein, for depth-penetrating modalities such as OCT, the image reconstruction algorithm 1000 may be applied to reconstruct a 3-dimensional volumetric view of the sample.

Additionally, the example shown in FIGS. 12-16 describe the reconstruction of an image as collected by a single-core OCT optical fiber 33. The image reconstruction algorithm 1000 may be extended for use with a multicore fiber 70. When utilizing a multicore fiber 70, each fiber core 71 will scan a different scanning pattern 60, based on its physical location within the fiber cladding 72 and providing a unique input stream Ii(t). In mapping a spirograph pattern 2000 based on multicore fiber, the pattern function F requires an additional variable, i, to distinguish the unique scanning patterns of each fiber core 71. When sorting the input from the multicore fiber 1002, each fiber core 71 will collect a unique sequence of voxels, therefore generating a unique sorted input I′i corresponding to each fiber core 71 with higher anticipated voxel density. In transforming the input 1100, all sorted inputs I′i(j,k,n) are mapped to a single transformed image V.

In use, including during system assembly and testing, the optical path may be debugged using a power throughput method. Debugging identifies dirty interfaces, broken fiber, components out of specification, and other issues which can impact the imaging process and output. Debugging also provides an estimate of the overall efficiency/throughput of the OCT system 100. The OCT system 100 is configured to limit power delivery to ensure that an upper bound on the power delivered to target tissue 200 is never exceeded.

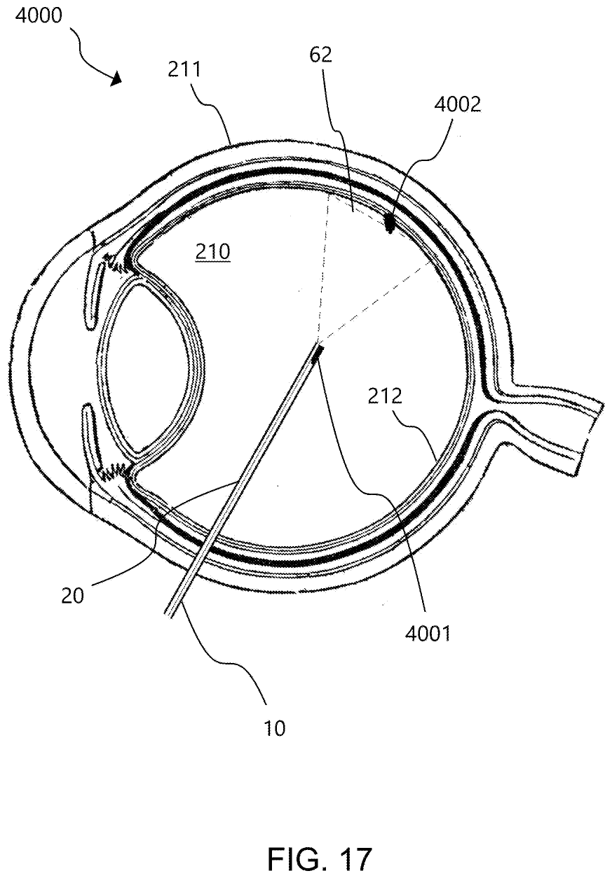

Referring now to FIG. 17, an embodiment of the inventive subject matter applied to the detection and removal of shrapnel from a subject's eye is shown. The inventive subject matter addresses a critical and urgent need to allow a physician to debride foreign objects 4002 such as shrapnel from the eyes 210 of soldiers suffering from blast injuries by using an approach that bypasses the cornea 211. The type of shrapnel debris may be organic, metallic (ferric or non-ferric), glass, or plastic. It is imperative to remove as much debris as possible, no matter how small, to avoid infection, fungal growth, scarring, and toxic reactions. Unless the blast injury is very fresh, vitrectomy, i.e., removal of all or part of the vitreous humor from the eye, must be employed to release each fragment from the vitreous humor before manual extraction using magnets, micro forceps, or a lasso. Extracting fragments otherwise will pull the vitreous humor, which is likely to cause retinal detachment, which severely impairs vision. Furthermore, debris may be in the retina 212 or near the retina 212 and thus, the retina 212 is at risk of injury from instrumentation used.

Existing clinical OCT machines can image the retina 212 only through the cornea, which is the transparent front part of the eye 210 that covers the iris, pupil, and anterior chamber of the eye 210. However, according to the inventive subject matter, the invention allows OCT to be introduced into the interior of the eye 210 via a trans-scleral penetration rather than through the cornea. Consequently, the eye 210 can be effectively and safely debrided of shrapnel or foreign objects 4002 while the cornea is left undisturbed. A three-dimensional microscopic surgical imaging instrument based upon the inventive subject matter described herein greatly increases the safety of the shrapnel extraction procedure and avoids collateral damage and additional recovery associated with working through or removing the cornea.

Microscopic imaging through a clear cornea offers the advantage of locating tiny fragments as compared to endoscopy. However, when the cornea is opaque, a hole must be made in the cornea to utilize microscopic imaging. This approach compromises the success of potential future corneal transplants that might be required to restore a soldier's vision. Further, if the cornea is destroyed due to an injury, trans-cornea imaging techniques do not work. As previously described, Optical Coherence Tomography (OCT) is an elegant photonic imaging modality that can produce microscopic images but is limited to surface imaging applications to detect and monitor anatomical anomalies and disease processes. In an OCT-assisted optometric procedure 4000 according to the inventive subject matter, hypotube insert 20 is deployed within imaging probe 10 and is then inserted into the interior of an individual's eye 210 through the sclera 211. Utilizing OCT as previously described, a physician can image the retina 212 at a desired focal plane 62 and detect a foreign object 4002 embedded in the retina 212. The imaging probe 10, in this case, a surgical needle, may also possess a removal tool 4001 such as a micro-forceps, lasso, or magnetic retrieval tool. The physician, relying on the OCT image for orientation, can navigate the imaging probe 10 to the foreign object 4002, acquire the foreign object 4002 using the removal tool 4001, and retract the imaging probe 10 while grasping the foreign object 4002 to remove the foreign object 4002 from the subject's eye 210. By penetrating the eye 210 via the sclera 211 rather than through the lens, the subject will experience less trauma and faster recovery time since the entry point of the imaging probe 10 will heal more readily than removal of a portion of the subject's lens.

Now referring to FIGS. 18A-18B, an additional exemplary embodiment of the inventive subject matter as applied to nerve ablation is shown. During a nerve ablation procedure 5000 according to the inventive subject matter described herein, the imaging probe 10 includes a surgical introducer needle 11 that is inserted subcutaneously into a patient's body tissue 200, such as in the proximity of the patient's spine 220, near a target nerve 221. The target nerve 221 is a nerve identified as likely requiring therapeutic ablation and may be near other biological structures 222, such as muscle tissue, fat tissue, bone, or vascular tissue, which are preferably not exposed to the ablative energy intended for application to the nerve 221. The surgical introducer needle 11, would likewise accommodate an ablation tool 5001, such as an ablative laser or an ablative radiofrequency transmitter. The OCT system 100 would provide an enface view from the surgical introducer needle 11 at a specific focal plane 62.

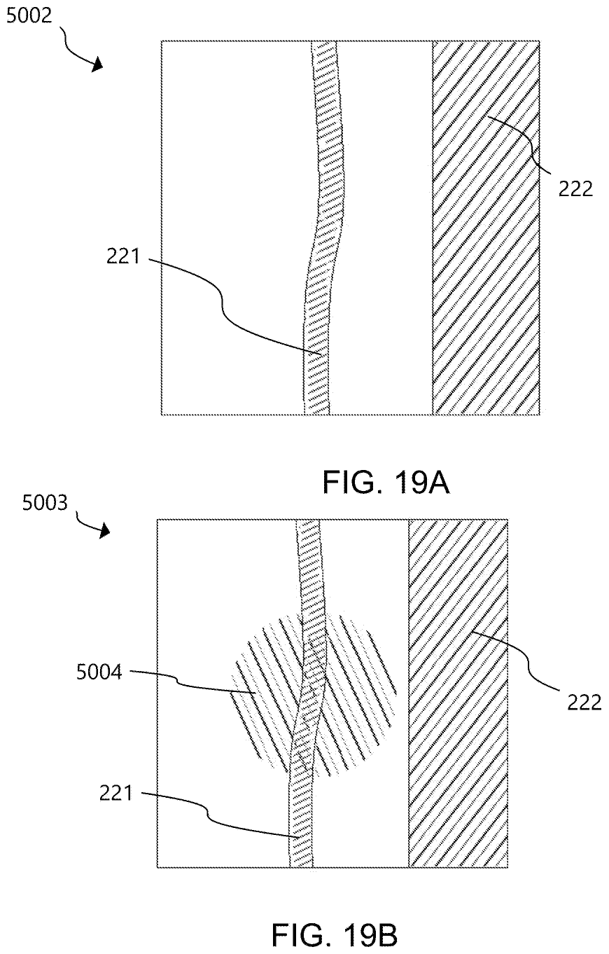

Referring now to FIGS. 19A-19B, the physician, relying on a pre-ablation OCT image 5002 for position and orientation, an example thereof shown in FIG. 19A, can navigate the surgical introducer needle 11 to the target nerve 221 and confirm that the ablation tool 5001 is appropriately directed to the target nerve 221 by, in one instance, centering the target nerve 221 in the focal plane 62. Furthermore, the physician may confirm that the ablation tool 5001 is properly oriented to minimize ablative damage to other biological structures 222. The physician may then ablate the target nerve 221 utilizing the ablation tool 5001.

In a first instance, the physician may use low power radiofrequency or laser application to diagnostically determine if the target nerve 221 is responsible for the patient's pain, which is enhanced by simultaneous imaging of the target tissue 200. Once the offending or responsible nerve 221 has been identified via the diagnostic procedures, the physician may then proceed to treat the nerve 221 with ablation or other therapeutics, e.g., anesthesia medications.

After ablation has been completed, the physician may inspect the ablated region 5004 and nerve 221 using a post-ablation OCT image 5003 created by the imaging probe 10, an example of the image thereof shown in FIG. 19B, to confirm whether the ablation was successful. Ablated tissue will typically develop differential optical properties as compared to that of the original non-ablated tissue. Thus, the ablated region 5004 would be differentially visible in the OCT image generated via probe 10 according to the inventive subject matter described herein. Thus, rather than relying solely on the subjective anecdotal response from a patient, and favorably avoiding the need to extend the lateral and volumetric range of the ablative area, the probe 10 allows a physician to perform the desired procedure more precisely and more directly confirm and validate its success or lack thereof. In addition, the differential visual properties of the ablated area can provide further information to the physician to determine what level of ablation occurred based on the areal extent, depth, color, and other lesion properties.

The ability to confirm successful ablation via post-procedure optical imaging afforded by the probe 10 allows the physician to provide confirming evidence of success or failure for subsequent review that has heretofore been unavailable. Thus, the physician can begin to adapt treatment procedures according to empirical data, rather than anecdotal data, to provide an appropriate level of ablation to achieve desired therapeutic results. Consequently, the physician can deliver more precise treatment with significantly more granularity across a broader treatment spectrum. Equally important is the ability to confidently confirm that a procedure, although successfully implemented, still failed to achieve the desired therapeutic results.

FIGS. 1 through 20, wherein like parts are designated by like reference numerals throughout, illustrate example embodiments of an OCT-enabled precision image-guided surgical probe according to the present invention. Although the present invention will be described with reference to the example embodiments illustrated in the figures, it should be understood that many alternative forms can embody the present invention. One of skill in the art will additionally appreciate diverse ways to alter the parameters of the embodiments disclosed, such as the size, shape, or type of elements or materials, in a manner still in keeping with the spirit and scope of the present invention.

We claim:

1. A precision image-guided surgical probe comprising:
a. a surgical introducer needle;
b. a dual parallel hypotube insert longitudinally enclosed within the surgical introducer needle;
c. the dual parallel hypotube insert having two hypotube working channels comprising:
(i) a fiber carrier; and
(ii) a therapeutic channel;
d. the fiber carrier and the therapeutic channel joined along their lengths at a seam such that the dual parallel hypotube insert may translate linearly and rotationally within the surgical introducer needle;
e. the fiber carrier having a lumen through which a fiber core is longitudinally disposed allowing linear translation and rotation of the fiber core to support optical imaging via a scanning pattern established by rotation of the dual parallel hypotube insert and rotation of the fiber core within the lumen and the application of optical coherence tomography; and
f. an annular space within the surgical introducer needle supporting delivery of fluid.

2. The precision image-guided surgical probe of claim 1, wherein a plurality of rotational speeds may be applied to the dual parallel hypotube insert and the fiber core in either clockwise or counter-clockwise directions.

3. The precision image-guided surgical probe of claim 1, wherein the precision image-guided surgical probe is configurable to provide improved precision neurosurgery to support precision treatment of nerves.

4. The precision image-guided surgical probe of claim 1, wherein the precision image-guided surgical probe facilitates precision injection of nerve modulating agents, the precise placement of microelectrodes for neuromodulation and neuro-stimulation, and the introduction of contrast.

5. The precision image-guided surgical probe of claim 1, wherein the fiber core is coupled to a fiber-optic slip ring to enable constant rotation of both the fiber core and the fiber carrier without damaging the fiber core.

6. The precision image-guided surgical probe of claim 1, wherein the fiber core comprises a multi-core fiber.

7. The precision image-guided surgical probe of claim 6, further comprising a fiber adapter and multiplexer to individually address each individual fiber core of the multi-core fiber to acquire optical signals simultaneously from each individual fiber core.

8. The precision image-guided surgical probe of claim 1, wherein the fiber core comprises a multi-core fiber having at least nineteen(should be 7??) cores on hexagonal stacking patterns.

9. A precision image-guided surgical probe comprising:
a. a surgical introducer needle having an interior;
b. an imaging insert housed inside the surgical introducer needle, the imaging insert linearly and rotationally translatable within the interior of the surgical introducer needle and having an interior wall;
c. the imaging insert comprising:
(i) a fiber carrier; and
(ii) a therapeutic channel having a lumen;
d. the therapeutic channel joined via a joining means along its length to the interior wall of the imaging insert such that the therapeutic channel translates in a circle about a center axis of the imaging insert as the imaging insert rotates;
e. the fiber carrier able to linearly translate and rotate independently of the interior wall of the imaging insert;
f. the fiber carrier including an optical fiber bonded to an interior of the fiber carrier such that the optical fiber is caused to rotate as the fiber carrier rotates;
g. the imaging insert and the therapeutic channel able to rotate in either a clockwise or a counter-clockwise direction as the fiber carrier rotates independently in either a clockwise or a counter-clockwise direction.

10. A precision image-guided surgical probe further according to claim 9, further comprising an annular space within the interior of the surgical introducer needle supporting delivery of fluid.

11. A method for delivering forward-looking precision imagery using a precision image guided surgical probe applying dual rotational scanning comprising:
a. deploying an imaging insert within an introducer needle such that the imaging insert may rotate in either a clockwise or a counterclockwise direction about a center axis of the imaging insert;
b. deploying an optical fiber within a lumen of an imaging fiber carrier such that the optical fiber will rotate in either a clockwise or counterclockwise direction about a center axis of the optical fiber;
c. polishing a tip of the optical fiber such that a laser light transmitted through the optical fiber exits from the tip of the optical fiber at an angle;
d. transmitting an optical coherence tomography beam through the optical fiber, the optical coherence tomography beam precessing in a spirograph scanning beam pattern to generate an acquired image;
e. directing the scanning beam pattern in a forward-looking manner, supporting acquisition of resolute tissue information and imagery while expanding the areal extent of the acquired image; and
f. varying the scanning beam pattern by varying rotational speed of the optical fiber and the imaging insert, thereby adapting a sampling density to suit specific tissue requirements or procedural requirements.

12. The method of claim 11, further comprising applying stereoscopic image acquisition with wearable and heads-up type displays for three-dimensional image processing.

13. The method of claim 11, further comprising delivering other tools through a therapeutic lumen to support performance of various surgical procedures in conjunction with forward-looking imaging.

14. A method for reconstructing an image of a target tissue from a surgical imaging probe, comprising:
a. selecting spirographic imaging parameters;
b. rotating an introducer needle around a first axis of rotation;
c. rotating an optical fiber around a second axis of rotation;
d. scanning a target tissue via the optical fiber;
e. sorting sensor input into individual frames;
f. transforming sensor input onto an image space according to spirographic imaging parameters,
g. interpolating missing input; and,
h. rendering a 2-dimensional image of the target tissue for display to a user.

15. The method of claim 14, wherein the scanning step is performed by an Optical Coherence Tomography imaging apparatus.

16. The method of claim 14, wherein the spirographic imaging parameters define a 2-dimensional, front-facing image space.

17. The method of claim 14, wherein the spirographic imaging parameters define a 3-dimensional, volumetric image space.

18. The method of claim 14, wherein the spirographic imaging parameters define a single core optical fiber geometry.

19. The method of claim 14, wherein the spirographic imaging parameters define a multicore optical fiber geometry.

20. The method of claim 14, wherein the interpolated missing input is defined using a nearest neighbor interpolation algorithm, a linear interpolation algorithm, a polynomial interpolation algorithm, a multi-variable interpolation algorithm, and a Gaussian process.
